SEQUENCE LISTING (6 sequences)

SEQ ID NO: 1 - protein, 22 aa, Artificial Sequence
  Synthetic construct
  C6-C22 disulfide
GLSKGCFGLKLDRIGSMSGLGC

SEQ ID NO: 2 - protein, 39 aa, Artificial Sequence
  Synthetic construct
  C23-C39 disulfide
PGQEHPNARKYKGANKKGLSKGCFGLKLDRIGSMSGLGC

SEQ ID NO: 3 - protein, 38 aa, Artificial Sequence
  Synthetic construct
  C22-C38 disulfide
LQEHPNARKYKGANKKGLSKGCFGLKLDRIGSMSGLGC

SEQ ID NO: 4 - protein, 38 aa, Artificial Sequence
  Synthetic construct
  C22-C38 disulfide
LQEHPQARKYKGAQKKGLSKGCFGLKLDRIGSMSGLGC

SEQ ID NO: 5 - protein, 38 aa, Artificial Sequence
  Synthetic construct
  C22-C38 disulfide
  VARIANT 33: Xaa = Nle
LQEHPQARKYKGAQKKGLSKGCFGLKLDRIGSXSGLGC

SEQ ID NO: 6 - protein, 39 aa, Artificial Sequence
  Synthetic construct
  C6-C22 disulfide
GLSKGCFGLKLDRIGSMSGLGCVQQRKDSKKPPAKLQPR

(19) United States
(12) Patent Application Publication
(10) Pub. No.: US 2024/0041982 A1
(43) Pub. Date: Feb. 8, 2024

(54) EXTENDED RELEASE HYDROGEL CONJUGATES OF C-NATRIURETIC PEPTIDES

(71) Applicant: ProLynx LLC, San Francisco, CA (US)

(72) Inventors: Eric L. SCHNEIDER, Oakland, CA (US); Brian R. HEARN, Moraga, CA (US); Daniel V. SANTI, San Francisco, CA (US)

(73) Assignee: ProLynx LLC, San Francisco, CA (US)

(21) Appl. No.: 18/038,687

(22) PCT Filed: Nov. 24, 2021
(86) PCT No.: PCT/US2021/060763

§ 371 Date: May 24, 2023

Related U.S. Application Data
(60) Provisional application No. 63/118,568, filed on Nov. 25, 2020.

(51) Int. Cl. A61K 38/22; A61K 47/69

(52) U.S. Cl. CPC A61K 38/2242; A61K 47/6903

(57) ABSTRACT

Provided herein are extended release hydrogel conjugates of c-natriuretic peptides, methods of preparation thereof, and methods of use thereof.

### CROSS-REFERENCE TO RELATED APPLICATION

This application claims priority to and the benefit of U.S. Provisional Application No. 63/118,568, filed on Nov. 25, 2020, the disclosure of which is incorporated herein by reference in its entirety.

### STATEMENT REGARDING SUBMISSION OF SEQUENCE LISTING

The present application is being filed along with a Sequence Listing in electronic format. The Sequence Listing is provided as a file entitled 670572002640SeqList.txt, created Nov. 19, 2021, which is 3,047 bytes in size. The information in the electronic format of the Sequence Listing is incorporated by reference in its entirety.

### FIELD

Provided herein are extended release hydrogel conjugates of c-natriuretic peptides, methods of preparation thereof, and methods of use thereof.

### BACKGROUND

C-type natriuretic peptide (CNP) is a member of a family of natriuretic peptides including atrial natriuretic peptide (ANP) and brain natriuretic peptide (BNP) that are released in response to hypertensive and hypervolaemic states, and promote natriuresis and diuresis resulting in loss of sodium and water thereby lowering blood volume and pressure. While CNP was first isolated in 1990 and was the last of the three main natriuretic peptides to be discovered, it is the most widely expressed of the family, being found especially in brain, chondrocytes, and endothelial cells. Normally acting as a local paracrine/autocrine regulator, exogenous CNP is a potent dilator of arteries and veins ex vivo, and has been shown to lower blood pressure in vivo in humans. Of the many non-cardiovascular effects, CNP has a primary role in the regulation of bone growth and may also play a role in neuronal development and protection. Genetic disruption of CNP production in mice leads to dwarfism through the reduction of bone length; femurs, tibiae, and vertebrae are 50-80% shorter than in wild-type mice. As a result, CNP and analogs have attracted attention for the treatment of dwarfism and achondroplasia.

Natriuretic peptides are characterized by a core 17-amino acid disulfide-linked ring that is critical for receptor binding. This cyclic structure is conserved across members of the family and between species. The initial product of the CNP gene (Nppc) is a 126-amino acid prepro-CNP; cleavage of a signal peptide yields a pro-CNP peptide that is further processed by the furin proprotein convertase to yield the 53-amino acid CNP-53. CNP-53 is further processed by as-yet unidentified proteases to provide the predominant active species, the 22-amino acid CNP (CNP-22). CNP activity is tightly regulated by two major degradative pathways resulting in an extremely short plasma half-life (˜3 minutes). CNP has high affinity for a clearance receptor, NPR-C, that internalizes the peptide into the lysosome for degradation. It is also proteolyzed by neutral endopeptidase (NEP) in plasma and on endothelial cell surfaces.

Three specific natriuretic peptide receptors have been characterized, NPR-A, NPR-B, and NPR-C. The low affinity of CNP for NPR-A suggests it may not be important for mediating physiological responses to CNP. CNP appears to be the sole endogenous ligand for NPR-B and can also bind and activate NPR-C. CNP binds NPR-B at physiological concentrations (picomolar) with an affinity 50-500 times greater than that of ANP and BNP. Genetic knock-out of NPR-B results in impairment of endochondrial ossification and resulting longitudinal shortening of vertebrae and limb bones. This model has further suggested a role for NPR-B in development of the female reproductive tract. NPR-B is found primarily on veins, but also on arteries. NPR-C binds all three natriuretic peptides with high affinity. Genetic knockout of NPR-C also results in skeletal abnormalities and increased basal bone turnover, presumably due to a shift in clearance of CNP from NPR-C to NPR-B.

Achondroplasia is a genetic disorder characterized by dwarfism caused by a mutation that results in an overactive fibroblast growth factor receptor 3 (FGFR3). It is the most prevalent form of dwarfism, affecting about 1 in 27,500 people. The predominant phenotype is short height, on average about 4 feet, an enlarged head and prominent forehead. Associated complications include sleep apnea, recurrent ear infections, obesity, hydrocephalus, and spinal stenosis. Growth hormone therapy is not effective for patients with achondroplasia. FGFR3 normally down-regulates cartilage and bone growth by inhibiting the development of chrondrocytes, cells that produce and maintain the cartilaginous matrix necessary for bone growth; hyperactive FGFR3 thus results in diminished bone growth and achondroplasia. Binding of finbroblast growth factors to FGFR3 results in a signaling cascade via the MAPK/ERK pathway. This cascade can be interrupted by activation of NPR-B, which interferes with the RAF-1 protein in the MAPK/ERK pathway. Thus, CNP or CNP analogs may find utility in the treatment of achondroplasia.

Several CNP analogs have been disclosed (U.S. Pat. Nos. 8,198,242, 8,377,884, and 9,266,939). A CNP analog having increased NEP resistance due to an increase in peptide chain length, vosoritide (BMN-111), has a somewhat-extended half-life (20 minutes) and has shown promise as a once-daily therapy for achondroplasia in Phase 3 clinical trials and is currently under review for approval at the FDA. Various analogs as well as PEGylated conjugates of CNP have also been investigated (Wendt, J Pharmacol Exp Ther 353:132-149, April 2015). Controlled-release conjugates of CNP have been disclosed (PCT Publication Nos. WO2016/110577, WO2017/118703, WO2017/118693, WO2017/118698, WO2017/118700, WO2017/118704, and WO2017/118707). Known side-effects of BMN-111 are increased heart rate and a drop in arterial blood pressure, which become more prominent as the dose is increased. The short half-life of BMN-111 requires a relatively high dose in order to provide therapeutic peptide levels for a sufficient time given a daily administration schedule. A significant increase in the half-life would allow for maintenance of therapeutic levels of peptide between dosings without the need for such over-dosing.

CNP prodrugs that extend the half-life of CNP through releasable conjugation have been disclosed (Breinholt et al., 2019 J. Pharmacol Exp Ther 370: 459-71; PCT Publication Nos. WO2016/110577, WO2017/118698, and WO2019/0022237). Combination therapy using controlled-release CNP analogs has also been disclosed (PCT Publication No. WO2018/060314). The disclosed conjugates release CNP through a hydrolytic mechanism, which is disadvantageous as it is difficult to avoid premature release from the conjugate during storage, leading to degradation and shortened shelf-life in the presence of moisture; this has necessitated development of dry formulations (PCT Publication No. WO2020/165081). Such formulations require reconstitution prior to use, however, and may not be suitable for insoluble conjugates such as microparticulate hydrogels. There thus remains a need for longer-acting forms of CNP in more convenient forms for the treatment of various conditions and diseases.

### BRIEF DESCRIPTION OF THE DRAWINGS

FIG. 1 illustrates two linker-CNPs of Formula (II). In both embodiments, Z=azide, n=1, R²=H, each R⁴=methyl, and E is [(Gln^{6,14})CNP38] attached to the linker via the alpha-amine of the N-terminal glycine. In the first linker-peptide, R¹=isopropyl-SO₂— and the linker releases the peptide with a half-life of 260 hours at pH 7.4, 37° C. after conjugation to hydrogel microspheres. In the second linker-peptide, R¹=(N.N-dimethylamino)-SO₂— and the linker releases the peptide with a half-life of 1200 hours at pH 7.4, 37° C. after conjugation to hydrogel microspheres.

FIG. 2 diagrams a method for producing a conjugate of Formula (IV) wherein M is a hydrogel comprising degradable crosslinks, comprising the steps of contacting a hydrogel of Formula (III) comprising a reactive connecting group Z′ with a linker-peptide of Formula (II) comprising a cognate reacting group Z under conditions wherein connecting functionality Z reacts with connecting functionality Z′ so as to conjugate the linker-peptide to the hydrogel through residual functionality Z*.

FIG. 3 illustrates the structures of crosslinks in conjugates of Formula (I) wherein M is an insoluble hydrogel, as illustrated in Example 3. In each case, the inter-polymer crosslink comprises linkers having B*=triazole, C*=carboxamide, q=1, R^{1a}=(N,N-dimethylamino)sulfonyl, R^{2a}=H, and R^{4a}=methyl. In the first case, a linker-CNP of Formula (II) wherein Z=azide, n=1, R¹=isopropyl-SO₂—, R²=H, each R⁴=methyl, and E is (Gln^{6,14})CNP38 attached to the linker via the alpha-amine of the N-terminal glycine has been conjugated to the hydrogel of Formula (III) having x=0, y=4, and z=0, through a carbamoyl-bicyclononynyl group. In the second case, a linker-CNP of Formula (II) wherein Z=azide, n=1, R¹=(N,N-dimethylamino)SO₂—, R²=H, each R⁴=methyl, and E is (Gln^{6,14})CNP38 attached to the linker via the alpha-amine of the N-terminal glycine has been conjugated to the hydrogel of Formula (III) having x=4, y=0, and z=0, through a carbamoyl-bicyclononynyl group.

FIG. 4 illustrates one method to prepare a linker-CNP of Formula (II) wherein the protected CNP peptide is prepared on a solid support using standard methods, the linker is attached by reaction with a succinimidyl carbonate, and the peptide is then deblocked, cleaved from the resin, and the disulfide is formed.

FIG. 5 illustrates an idealized structure showing the disposition of P¹ (filled circles) and P² (open circles) and linker-drug L-E (black circles) in the crosslinked matrix M. The two polymers alternate in the matrix due to their connection via cognate groups Z and Z′, which prevents self-connection, and each crosslink comprises a linker-drug. In practice, some crosslinks may be missing, for example due to missing arms in commercial preparations of the polymers or due to formation of multiple crosslinks between individual P¹ and P² units.

FIG. 6 shows the results of the stability study of Example 1. Vosoritide and (Gln⁶,Gln¹⁴)CNP38 were kept at pH 7.4, 37° C. and monitored by anion-exchange HPLC. (Gln⁶,Gln¹⁴)CNP38 showed enhanced stability over vosoritide.

FIGS. 7A-7B show the results of pharmacokinetic experiments in mice treated with the conjugates of Example 3 (also FIG. 3). Panel A shows the concentration of (Gln^{6,14})CNP38 in the plasma of mice after subcutaneous injection of the conjugate of Example 3 wherein R¹=isopropylsulfonyl. Panel B shows the concentration of (Gln^{6,14})CNP38 in the plasma of mice after subcutaneous injection of the conjugate of Example 3 wherein R¹=(N,N-dimethylamino)sulfonyl.

FIG. 8 shows the results of pharmacokinetic experiments in juvenile cynomolgous monkeys treated with the conjugates of Example 3 wherein R¹=isopropylsulfonyl. FIG. 8 depicts plasma concentrations of [Gln6,14]CNP-38 after a single subcutaneous administration of 1.3 umol/kg of the conjugate of Example 3 wherein R¹=isopropylsulfonyl to juvenile cynomolgous monkeys. Data are averages of 3 animals. This conjugate provided continuous exposure ≥100 pM of [Gln6,14]CNP-38 for approximately 1 month.

FIG. 9 shows the results of pharmacokinetic experiments in juvenile cynomolgous monkeys treated with the conjugates of Example 3 wherein R¹=(N,N-dimethylamino)sulfonyl. plasma concentrations of [Gln^{6,14}]CNP-38 after a single subcutaneous administration of 1.2 umol/kg of the conjugate of Example 3 wherein R¹=(N,N-dimethylamino)sulfonyl to juvenile cynomolgous monkeys. Data are averages of 3 animals. This conjugate provided continuous exposure ≥100 pM of [Gln^{6,14}]CNP-38 for greater than 3 months.

FIG. 10 shows the results of measurements of total length (tail length+naso-anal length, TL) of mice treated with [Gln6,14]CNP-38 and the conjugates of Example 3 wherein R¹=isopropylsulfonyl (4A) or (R¹=(N,N-dimethylamino)sulfonyl (4B) at constant amount per mouse each dosing interval. From left to right: black (solid), vehicle control; brown (empty), QD [Gln^{6,14}]CNP-38; blue (horizontal stripe), QWk 4A 20 nmol/mouse; red (rising diagonal), QWk 4A 50 nmol/mouse; green (falling diagonal), single dose 4B 85 nmol/mouse. Plotted as percent of initial total length (TL) vs time; values are displayed as the mean±SD.

FIG. 11 shows the results of measurements of total length (Tail length+Anal-Nasal length, TL) of mice treated with [Gln^{6,14}]CNP-38 and the conjugates of Example 3 wherein R¹=isopropylsulfonyl (4A) or (R¹=(N,N-dimethylamino)sulfonyl (4B) using weight-adjusted dosing (μmol/kg). From left to right, black (solid), vehicle control; brown (empty), QD [Gln^{6,14}]CNP-38; blue (horizontal), QWk 4A 1.5 μmol/kg; red (rising diagonal), QWk 4A 2.2 μmol/kg; green (falling diagonal), Q2Wk 2.2 μmol/kg; purple (checkered), single dose 4B 6.1 μmol/kg; orange (vertical), single dose 4B 40 μmol/kg. Plotted as percent of initial total length (TL) vs time; values are displayed as the mean±SD.

FIG. 12 shows a photograph of representative mice treated with [Gln^{6,14}]CNP-38 or the conjugate 4A (Example 3 wherein R¹=isopropylsulfonyl) for five weeks. A) vehicle control; B) QD [Gln^{6,14}]CNP-38 peptide at 70 nmol; C) biweekly (Q2Wk) conjugate 4A at 2.2 μmol/kg; D) weekly (QWk) conjugate 4A at 2.2 μmol/kg; and E) weekly (QWk) conjugate 4A at 1.5 μmol/kg. Anesthetized mice were initially positioned for measurements with their heads extended, noses aligned to the horizontal guide line and tails pulled straight; the distance between the top guide line and the end of the tail best represents total length (TL).

FIGS. 13A and 13B depict peptide release and hydrogel degradation of the conjugate of Example 3 wherein R¹=isopropylsulfonyl (FIG. 13A) and wherein R¹=(N,N-dimethylamino)sulfonyl (FIG. 13B) at pH 9.4, 37° C. In FIG. 13A, for R¹=isopropylsulfonyl, total solubilized peptide (solid line) and solubilized PEG (dashed line) gave a (Gln^{6,14})CNP38 release t_{1/2}=6.1 h, corresponding to 610 h under physiological conditions (pH 7.4, 37° C.). In FIG. 13B, for R¹=(N,N-dimethylamino)sulfonyl, total solubilized peptide (solid line) and solubilized PEG (dashed line) gave a (Gln^{6,14})CNP38 release t_{1/2}=15.8 h, corresponding to 1580 h under physiological conditions (pH 7.4, 37° C.). Data are the averages of n=6, with error bars given as standard deviations.

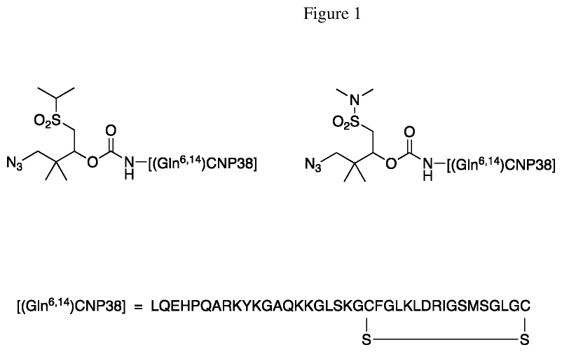

### DETAILED DESCRIPTION

The present disclosure is directed to conjugates that provide extended, low-level release of C-natriuretic peptides (CNPs) that support once-weekly, once-monthly, or even less frequent administration of these peptides and are expected to be useful in the treatment of diseases and conditions such as achondroplasia.

In one aspect, the present disclosure provides extended release conjugates comprising an insoluble hydrogel matrix with a multiplicity of covalently attached linker-peptides, wherein the linkers cleave via a beta-elimination mechanism under physiological conditions of pH and temperature to release free CNP peptides. The conjugates of the invention can be illustrated schematically as formula (I)

M-(L-E)ₐ (I)

wherein M is an insoluble hydrogel matrix connected to a multiplicity of CNP peptides E through cleavable linker L, L is a linker that cleaves by a pH-dependent beta-elimination mechanism, such as a linker as disclosed in U.S. Pat. No. 8,680,315, and a is an integer that represents the number of L-E moieties that yield a suitable concentration of E in a given volume of the matrix. Suitable concentrations are 0.01-50 mg peptide per mL of matrix, preferably 1-25 mg of peptide per mL. The linker L releases free CNP peptides with a half-life suitable for the desired period of administration, typically between 150 and 2500 hours as measured in vitro, preferably between 250 and 1500 hours in vitro at pH 7.4, 37° C.

In a second aspect, the present invention provides linker-CNP (L-E) having the formula (II)

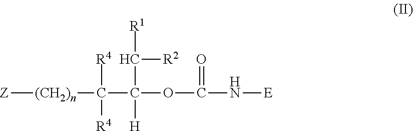

wherein:
n=0-6;
R¹ and R² are independently H, alkyl, CN, NO₂, optionally substituted aryl, optionally substituted heteroaryl, optionally substituted alkenyl, optionally substituted alkynyl, —COR³, —SOR³, or —SO₂R³, wherein
   R³ is H, optionally substituted alkyl, optionally substituted aryl, optionally substituted arylalkyl, optionally substituted heteroaryl, optionally substituted heteroarylalkyl, —OR⁵, or —NR⁵₂, wherein
      each R⁵ is independently H or optionally substituted alkyl, or both R⁵ groups are taken together with the nitrogen to which they attach to form a heterocyclic ring;
wherein R¹ and R² may be taken together with the carbon to which they attach to form a 3-8 membered ring, and wherein one and only one of R¹ and R² may be H or alkyl;
each R⁴ is independently H or C₁-C₃ alkyl, or both R⁴ are taken together with the carbon to which they attach to form a 3-6 membered ring;
Z is a functional group for mediating coupling to the carrier M through a cognate functional group Z′; and
NH is the residue of an amino group of CNP peptide E.

It is understood that the term “alkyl” includes linear, branched, or cyclic saturated hydrocarbon groups of 1-20, 1-12, 1-8, 1-6, or 1-4 carbon atoms. In some embodiment, an alkyl is linear or branched. Examples of linear or branched alkyl groups include, without limitation, methyl, ethyl, n-propyl, isopropyl, n-butyl, t-butyl, isobutyl, sec-butyl, n-pentyl, n-hexyl, n-heptyl, n-octyl, n-nonyl, n-decyl, and the like. In some embodiments, an alkyl is cyclic. Examples of cyclic alkyl groups include, without limitation, cyclopropyl, cyclobutyl, cyclopentyl, cyclopentadienyl, cyclohexyl, and the like.

It is understood that the term “alkoxy” includes alkyl groups bonded to oxygen, including methoxy, ethoxy, isopropoxy, cyclopropoxy, cyclobutoxy, and the like.

It is understood that the term “alkenyl” includes non-aromatic unsaturated hydrocarbons with carbon-carbon double bonds and 2-20, 2-12, 2-8, 2-6, or 2-4 carbon atoms.

It is understood that the term “alkynyl” includes non-aromatic unsaturated hydrocarbons with carbon-carbon triple bonds and 2-20, 2-12, 2-8, 2-6, or 2-4 carbon atoms.

It is understood that the term “aryl” includes aromatic hydrocarbon groups of 6-18 carbons, preferably 6-10 carbons, including groups such as phenyl, naphthyl, and anthracenyl. The term “heteroaryl” includes aromatic rings comprising 3-15 carbons containing at least one N, O or S atom, preferably 3-7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and the like.

In some instances, alkenyl, alkynyl, aryl or heteroaryl moieties may be coupled to the remainder of the molecule through an alkyl linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

It is understood that the term “halogen” or “halo” includes bromo, fluoro, chloro and iodo.

It is understood that the term “heterocyclic ring” or “heterocyclyl” refers to a 3-15 membered aromatic or non-aromatic ring comprising at least one N, O, or S atom. Examples include, without limitation, piperidinyl, piperazinyl, tetrahydropyranyl, pyrrolidine, and tetrahydrofuranyl, as well as the exemplary groups provided for the term “heteroaryl” above. In some embodiments, a heterocyclic ring or heterocyclyl is non-aromatic. In some embodiments, a heterocyclic ring or heterocyclyl is aromatic.

It is understood that “optionally substituted,” unless otherwise specified, means that a group may be unsubstituted or substituted by one or more (e.g., 1, 2, 3, 4 or 5) of the substituents which may be same or different. Examples of substituents include, without limitation, alkyl, alkenyl, alkynyl, halogen, —CN, —OR^{aa}, —SR^{aa}, —NR^{aa}R^{bb}, —NO₂, —C═NH(OR^{aa}), —C(O)R^{aa}, —OC(O)R^{aa}, —C(O)OR^{aa}, —C(O)NR^{aa}R^{bb}, —OC(O)NR^{aa}R^{bb}, —NR^{aa}C(O)R^{bb}, —NR^{aa}C(O)OR^{bb}, —S(O)R^{aa}, —S(O)₂R^{aa}, —NR^{aa}S(O)R^{bb}, —C(O)NR^{aa}S(O)R^{bb}, —NR^{aa}S(O)₂R^{bb}, —C(O)NR^{aa}S(O)₂R^{bb}, —S(O)NR^{aa}R^{bb}, —S(O)2NR^{aa}R^{bb}, —P(O)(OR^{aa}) (OR^{bb}), heterocyclyl, heteroaryl, or aryl, wherein the alkyl, alkenyl, alkynyl, cycloalkyl, heterocyclyl, heteroaryl, and aryl are each independently optionally substituted by R^{cc}, wherein
R^{aa} and R^{bb} are each independently H, alkyl, alkenyl, alkynyl, heterocyclyl, heteroaryl, or aryl, or
R^{aa} and R^{bb} are taken together with the nitrogen atom to which they attach to form a heterocyclyl, which is optionally substituted by alkyl, alkenyl, alkynyl, halogen, hydroxyl, alkoxy, or —CN, and wherein:
each R^{cc} is independently alkyl, alkenyl, alkynyl, halogen, heterocyclyl, heteroaryl, aryl, —CN, or —NO₂.

For use herein, unless clearly indicated otherwise, use of the terms “a”, “an” and the like refers to one or more.

The rate of drug release is tunable by the appropriate choice of groups R¹ and R². Descriptions of suitable R¹ and R² can be found in U.S. Pat. No. 8,680,315. In some embodiments, each of groups R¹ and R² may be independently substituted by electron-donating and/or electron-withdrawing substituents that alter the acidity of the intervening R¹R²CH proton so that enormous flexibility and control over the rate of drug elimination can be achieved. Electron-withdrawing groups are defined as groups having a Hammett sigma value greater than 0 (see, for example, Hansch et al. 1991 Chemical Reviews 91: 165-195). By the term “electron-donating group” is meant a substituent resulting in a decrease in the acidity of the R¹R²CH; electron-donating groups are typically associated with negative Hammett σ or Taft σ* constants and are well-known in the art of physical organic chemistry. (Hammett constants refer to aryl/heteroaryl substituents, Taft constants refer to substituents on non-aromatic moieties.) Examples of suitable electron-donating substituents include but are not limited to lower alkyl, lower alkoxy, lower alkylthio, amino, alkylamino, dialkylamino, and silyl. Similarly, by “electron-withdrawing group” is meant a substituent resulting in an increase in the acidity of the R¹R²CH group; electron-withdrawing groups are typically associated with positive Hammett σ or Taft σ* constants and are well-known in the art of physical organic chemistry. A description of suitable electron-donating and electron-withdrawing substituents that can be used to modulate R¹ and R² can be can be found in U.S. Pat. No. 9,649,385.

In some embodiments, at least one of R¹ and R² is —CN. In some embodiments, at least one of R¹ and R² is —NO₂. In some embodiments, at least one of R¹ and R² is optionally substituted aryl containing 6-10 carbons. For instance, in some embodiments, at least one of R¹ and R² is phenyl, naphthyl, or anthracenyl, each of which is optionally substituted. In some embodiments, at least one of R¹ and R² is optionally substituted heteroaryl comprising 3-7 carbons and containing at least one N, O, or S atom. For instance, in some embodiments, at least one of R¹ and R² is pyrrolyl, pyridyl, pyrimidinyl, imidazolyl, oxazolyl, isoxazolyl, thiazolyl, isothiazolyl, quinolyl, indolyl, or indenyl, each of which is optionally substituted. In some embodiments, at least one of R¹ and R² is optionally substituted alkenyl containing 2-20 carbon atoms. In some embodiments, at least one of R¹ and R² is optionally substituted alkynyl containing 2-20 carbon atoms. In some embodiments, at least one of R¹ and R² is —COR³, —SOR³, or —SO₂R³, wherein R³ is H, optionally substituted alkyl containing 1-20 carbon atoms, optionally substituted aryl, optionally substituted arylalkyl, optionally substituted heteroaryl, optionally substituted heteroarylalkyl, —OR⁵ or —NR⁵₂, wherein each R⁵ is independently H or optionally substituted alkyl containing 1-20 carbon atoms, or both R⁵ groups are taken together with the nitrogen to which they are attached to form a heterocyclic ring.

In some embodiments, at least one of R¹ and R² is —CN, —SOR³ or —SO₂R³. In some embodiments, at least one of R¹ and R² is —CN or —SO₂R³. In some embodiments, at least one of R¹ and R² is —CN or —SO₂R³, wherein R³ is optionally substituted alkyl, optionally substituted aryl, or —NR⁵₂. In some embodiments, at least one of R¹ and R² is —CN, —SO₂N(CH₃)₂, —SO₂CH₃, —SO₂Pherlyl, —SO₂(chlorophenyl), —SO₂(4-methylphenyl), —SO₂N(CH₂CH₂)₂O, —SO₂N(CH₂CH₂)₂S, —SO₂CH(CH₃)₂, —SO₂N(CH₃)(CH₂CH₃), or —SO₂N(CH₂CH₂OCH₃)₂. In some embodiments, one of R¹ and R² is —CN or —SO₂R³, wherein R³ is optionally substituted alkyl, optionally substituted aryl, or —NR⁵₂; and the other is H. In some embodiments, one of R¹ and R² is —CN, —SO₂N(CH₃)₂, —SO₂CH₃, —SO₂Phenyl, —SO₂(chlorophenyl), —SO₂(4-methylphenyl), —SO₂N(CH₂CH₂)₂O, —SO₂N(CH₂CH₂)₂S, —SO₂CH(CH₃)₂, —SO₂N(CH₃)(CH₂CH₃), or —SO₂N(CH₂CH₂OCH₃)₂; and the other is H.

In some embodiments, each R⁴ is independently C₁-C₃ alkyl. In some embodiments, both R⁴ are methyl.

In some embodiments, n is an integer from 1 to 6. In some embodiments, n is an integer from 1 to 3. In some embodiments, n is an integer from 0 to 3. In some embodiments, n is 1. In some embodiments, n is 2. In some embodiments, n is 3. In some embodiments, n is 4. In some embodiments, n is 5. In some embodiments, n is 6.

In some embodiments, R¹ is CN or SO₂R³ as defined above, R² is H, each R⁴ is C₁-C₃ alkyl, and n=1-3.

Connecting group Z may be any group capable of selective reaction in the presence of a CNP peptide. Typical groups include, without limitation, azide, in which case cognate group Z′ on M is an alkyne or cycloalkyne and the residual connecting group is a triazole; aminoether, in which case cognate group Z′ on M is a carbonyl and the residual connecting group is an oxime; trans-cyclooctene or cyclopropane, in which case cognate group Z′ on M is a 1,2,5,6-tetrazine and the residual functionality is a pyridazine; or a thiol, in which case cognate group Z′ on M is a maleimide or halocarbonyl and the residual connecting group is a thioether. In preferred embodiments, Z is azide and Z′ is a cyclooctyne. Typical cyclooctynes known in the art include, without limitation, 5-hydroxycyclooctyne (5HCO), 1-fluorocyclooct-2yne-1-carboxylate (MFCO), bicyclo[6.1.0]non-4-yne (BCN) (see Dommerholt et al., Top Curr Chem (Z) (2016) 374:16).

The term “CNP” includes all peptides characterized by the ability to bind NPR-B and thereby regulate the growth, proliferation, and differentiation of chondrocytes. This includes, without limitation, sequences listed in PCT Publications 2009/067639, 2010/135541, and 2016/110577; and Morozumi et al. (2019) PLoS ONE 14(2): e0212680. Preferably, the term “CNP” refers to a peptide of SEQ ID No: 1-6 and stabilized analogs thereof. Particularly preferred are CNP analogs wherein particular amino acid residues have been replaced so as to improve the stability of the peptide. Such stabilized analogs include peptides wherein asparagine residues have been replaced by residues less susceptible towards deamidation, for example glutamine or alanine, and peptides wherein oxidation-sensitive residues have been replaced, for example methionine replaced by norleucine. Exemplary embodiments of CNP are provided in SEQ ID Nos: 1-6 below.

SEQ ID No: 1
([CNP22])
GLSKGCFGLKLDRIGSMSGLGC C⁶-C²² disulfide
SEQ ID No: 2
([vosoritide])
PGQEHPNARKYKGANKKGLSKGCFGLKLDRIGSMSGLGC C²³-C³⁹
disulfide
SEQ ID No: 3
([CNP38])
LQEHPNARKYKGANKKGLSKGCFGLKLDRIGSMSGLGC C²²-C³⁸
disulfide
SEQ ID No: 4
([(Gln^{6,14})CNP38])
LQEHPQARKYKGAQKKGLSKGCFGLKLDRIGSMSGLGC C²²-C³⁸
disulfide
SEQ ID No: 5
([(Gln^{6,14},Nle³³)CNP38])
LQEHPQARKYKGAQKKGLSKGCFGLKLDRIGSXSGLGC C²²-C³⁸
disulfide
SEQ ID No: 6
([ASB20123])
GLSKGCFGLKLDRIGSMSGLGCVQQRKDSKKPPAKLQPR C⁶-C²²
disulfide

In some embodiments, the stabilized CNP is (Gln^{6,14})-CNP38 (SEQ ID No: 4) or (Gln^{6,14},Nle³³)CNP38 (SEQ ID No: 5).

In the descriptions herein, it is understood that every description, variation, embodiment or aspect of a moiety may be combined with every description, variation, embodiment or aspect of other moieties the same as if each and every combination of descriptions is specifically and individually listed. For example, every description, variation, embodiment or aspect provided herein with respect to n of formula (II) may be combined with every description, variation, embodiment or aspect of R¹, R², R⁴, Z, and E, the same as if each and every combination were specifically and individually listed. It is also understood that all descriptions, variations, embodiments or aspects of any formulae such formula (I), (II), (III), (IV), or (V), where applicable, apply equally to other formulae detailed herein, and are equally described, the same as if each and every description, variation, embodiment or aspect were separately and individually listed for all formulae.

In some embodiments, M is a water-insoluble hydrogel carrier. In preferred embodiments, M is a degradable hydrogel of formula (III),

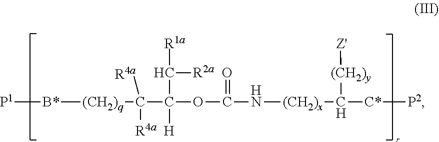

wherein q=0-6;
R^{1a} and R^{2a} are independently H, alkyl, CN, NO₂, optionally substituted aryl, optionally substituted heteroaryl, optionally substituted alkenyl, optionally substituted alkynyl, —COR^{3a}, —SOR^{3a}, or —SO₂R^{3a}, wherein
   R^{3a} is H, optionally substituted alkyl, optionally substituted aryl, optionally substituted arylalkyl, optionally substituted heteroaryl, optionally substituted heteroarylalkyl, —OR^{5a}, or —NR^{5a}₂, wherein
      each R^{5a} is independently H or optionally substituted alkyl, or both R^{5a} groups are taken together with the nitrogen to which they attach to form a heterocyclic ring;
wherein R^{1a} and R^{2a} may be taken together with the carbon to which they attach to form a 3-8 membered ring, and wherein one and only one of R^{1a} and R^{2a} may be H or alkyl;
each R^{4a} is independently H or C₁-C₃ alkyl, or both R^{4a} are taken together with the carbon to which they attach to form a 3-6 membered ring;
Z′ is a functional group for mediating coupling to cognate functional group Z of a linker-CNP of Formula (II) as disclosed herein;
x and y are each independently 0-6;
B* and C* are each independently a connecting group; and
P¹ and P² are independently r-armed polymers of 1-40 kDa average molecular weight, wherein r is an integer from 2 to 8. In preferred embodiments, P¹ and P² are r-armed poly(ethylene glycols).

The rate of degradation of the hydrogel of formula (III) is tunable by the appropriate choices of groups R^{1a} and R^{2a}, as discussed herein for R¹ and R².

In some embodiments, at least one of R^{1a} and R^{2a} is —CN. In some embodiments, at least one of R^{1a} and R^{2a} is —NO₂. In some embodiments, at least one of R^{1a} and R^{2a} is optionally substituted aryl containing 6-10 carbons. For instance, in some embodiments, at least one of R^{1a} and R^{2a} is phenyl, naphthyl, or anthracenyl, each of which is optionally substituted. In some embodiments, at least one of R^{1a} and R^{2a} is optionally substituted heteroaryl comprising 3-7 carbons and containing at least one N, O, or S atom. For instance, in some embodiments, at least one of R^{1a} and R^{2a} is pyrrolyl, pyridyl, pyrimidinyl, imidazolyl, oxazolyl, isoxazolyl, thiazolyl, isothiazolyl, quinolyl, indolyl, or indenyl, each of which is optionally substituted. In some embodiments, at least one of R^{1a} and R^{2a} is optionally substituted alkenyl containing 2-20 carbon atoms. In some embodiments, at least one of R^{1a} and R^{2a} is optionally substituted alkynyl containing 2-20 carbon atoms. In some embodiments, at least one of R^{1a} and R^{2a} is —COR^{3a}, —SOR^{3a}, or —SO₂R^{3a}, wherein R^{3a} is H, optionally substituted alkyl containing 1-20 carbon atoms, optionally substituted aryl, optionally substituted arylalkyl, optionally substituted heteroaryl, optionally substituted heteroarylalkyl, —OR^{5a} or —NR^{5a}₂, wherein each R^{5a} is independently H or optionally substituted alkyl containing 1-20 carbon atoms, or both R^{5a} groups are taken together with the nitrogen to which they are attached to form a heterocyclic ring.

In some embodiments, at least one of R^{1a} and R^{2a} is —CN, —SOR^{3a} or —SO₂R^{3a}. In some embodiments, at least one of R^{1a} and R^{2a} is —CN or —SO₂R^{3a}. In some embodiments, at least one of R^{1a} and R^{2a} is —CN or —SO₂R^{3a}, wherein R^{3a} is optionally substituted alkyl, optionally substituted aryl, or —NR^{5a}₂. In some embodiments, at least one of R^{1a} and R^{2a} is —CN, —SO₂N(CH₃)₂, —SO₂CH₃, —SO₂Phenyl, —SO₂(chlorophenyl), —SO₂(4-methylphenyl), —SO₂N(CH₂CH₂)₂O, —SO₂N(CH₂CH₂)₂S, —SO₂CH(CH₃)₂, —SO₂N(CH₃)(CH₂CH₃), or —SO₂N(CH₂CH₂OCH₃)₂. In some embodiments, one of R^{1a} and R^{2a} is —CN or —SO₂R^{3a}, wherein R^{3a} is optionally substituted alkyl, optionally substituted aryl, or —NR^{5a}₂; and the other is H. In some embodiments, one of R^{1a} and R^{2a} is —CN, —SO₂N(CH₃)₂, —SO₂CH₃, —SO₂Phenyl, —SO₂(chlorophenyl), —SO₂(4-methylphenyl), —SO₂N(CH₂CH₂)₂O, —SO₂N(CH₂CH₂)₂S, —SO₂CH(CH₃)₂, —SO₂N(CH₃)(CH₂CH₃), or —SO₂N(CH₂CH₂OCH₃)₂; and the other is H.

In some embodiments, each R^{4a} is independently C₁-C₃ alkyl. In some embodiments, both R^{4a} are methyl.

In some embodiments, q is an integer from 1 to 6. In some embodiments, q is an integer from 2 to 3. In some embodiments, q is an integer from 1 to 3. In some embodiments, q is an integer from 0 to 3. In some embodiments, q is 1. In some embodiments, q is 2. In some embodiments, q is 3. In some embodiments, q is 4. In some embodiments, q is 5. In some embodiments, q is 6.

In some embodiments, x is an integer from 1 to 6. In some embodiments, x is an integer from 2 to 3. In some embodiments, x is an integer from 1 to 3. In some embodiments, x is an integer from 0 to 3. In some embodiments, x is 1. In some embodiments, x is 2. In some embodiments, x is 3. In some embodiments, x is 4. In some embodiments, x is 5. In some embodiments, x is 6.

In some embodiments, y is an integer from 1 to 6. In some embodiments, y is an integer from 2 to 3. In some embodiments, y is an integer from 1 to 3. In some embodiments, y is an integer from 0 to 4. In some embodiments, y is 1. In some embodiments, y is 2. In some embodiments, y is 3. In some embodiments, y is 4. In some embodiments, y is 5. In some embodiments, y is 6.

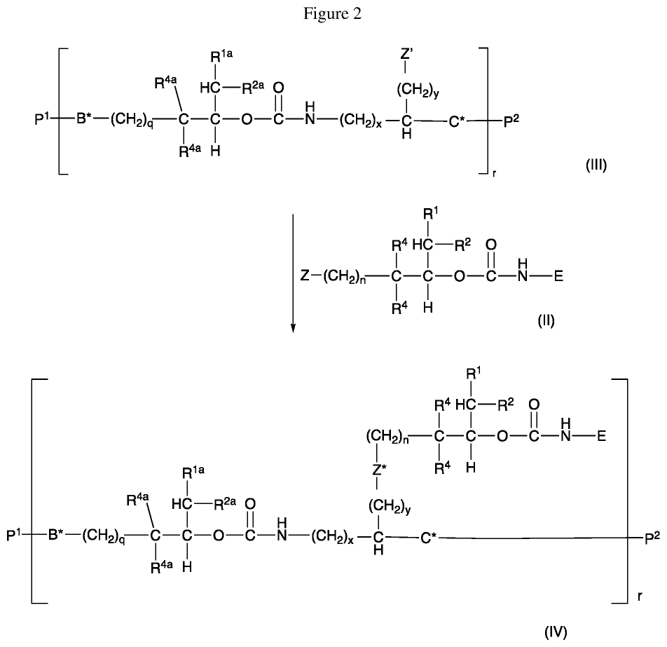

Hydrogels of Formula (III) where in P¹ and P² are r-armed poly(ethylene glycols) are prepared as described in, for example, Henise et al. (2015) Bioconj. Chem. 26: 270-8; Henise et al., Int. J. Polymer Sci. Vol. 2019, Article ID 9483127; and Henise et al. (2020) Engineering Reports 2020; 2:e12213. These hydrogels provide for tuned rates of drug release and subsequent hydrogel dissolution. Typical connecting groups of B* and C* include, without limitation, triazole, carboxamide, carbamate, oxime, and thioether. When hydrogel polymerization is by azide/cyclooctyne cycloaddition, the connecting group of B* and/or C* is triazole as described above. In some embodiments, B* is triazole and C* is carboxamide. P¹ and P² are synthetic or natural polymers such as poly(ethylene glycols), dextrans, hyaluronic acids, and the like. In these hydrogels, the polymer chains are crosslinked to form an insoluble 3-dimensional matrix (FIG. 6), where each crosslink has an attachment point for a linker-CNP of Formula (II). The crosslinks slowly cleave by non-hydrolytic beta-elimination at rates governed primarily by groups R^{1a} and R^{2a} to give ultimately soluble polymer fragments. These hydrogels allow for attachment of the linker-drugs via connecting group Z*, formed by reaction of cognate groups Z and Z′ on Formulas (II) and (III) as illustrated in FIG. 2, thus producing a conjugate of Formula (I) wherein M is an insoluble hydrogel comprising a conjugated linker-peptide at each crosslink and having the more specific Formula (IV), wherein R¹, R², R⁴, E, n, Z*, y, P¹, P², B*, q, r, C*, x, R^{1a}, R^{2a}, and R^{4a} are as disclosed herein.

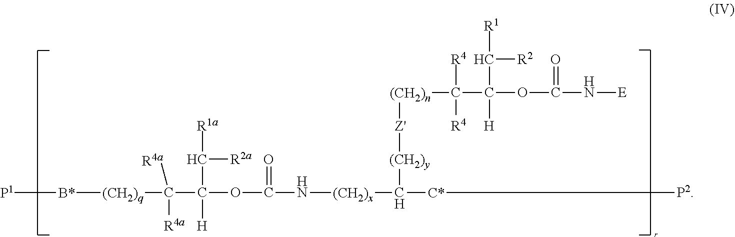

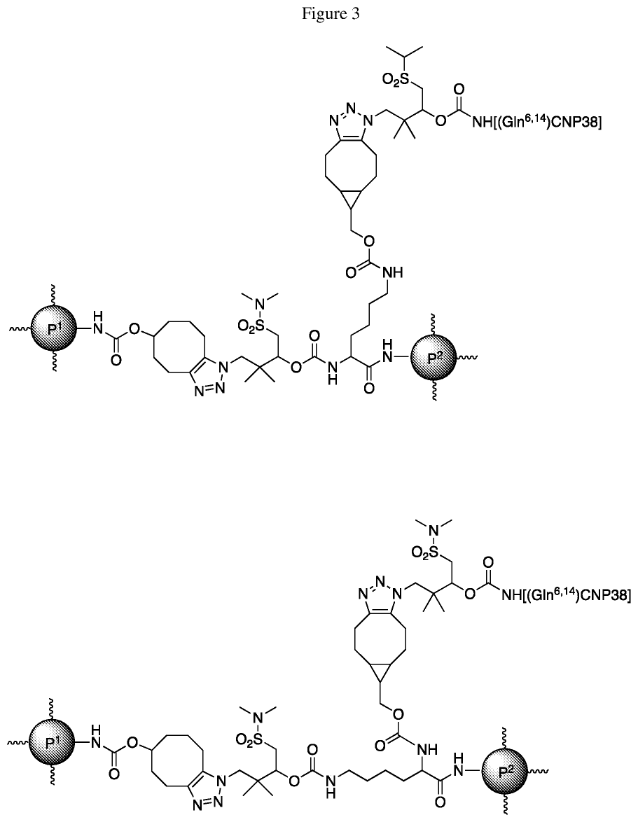

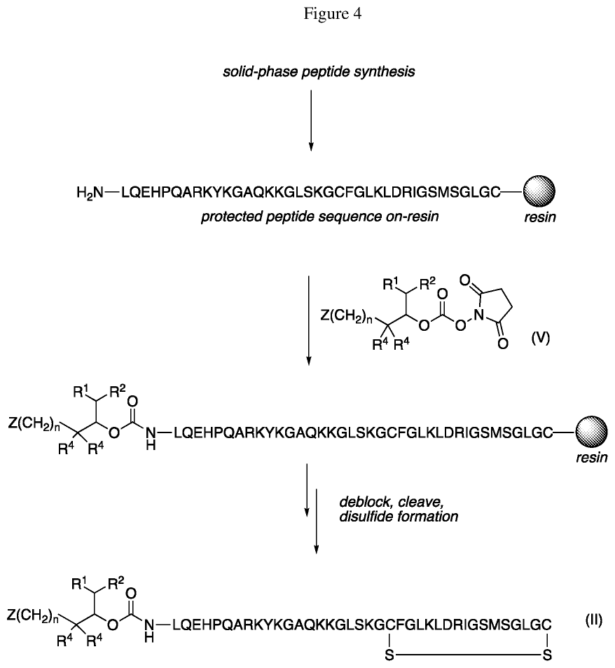

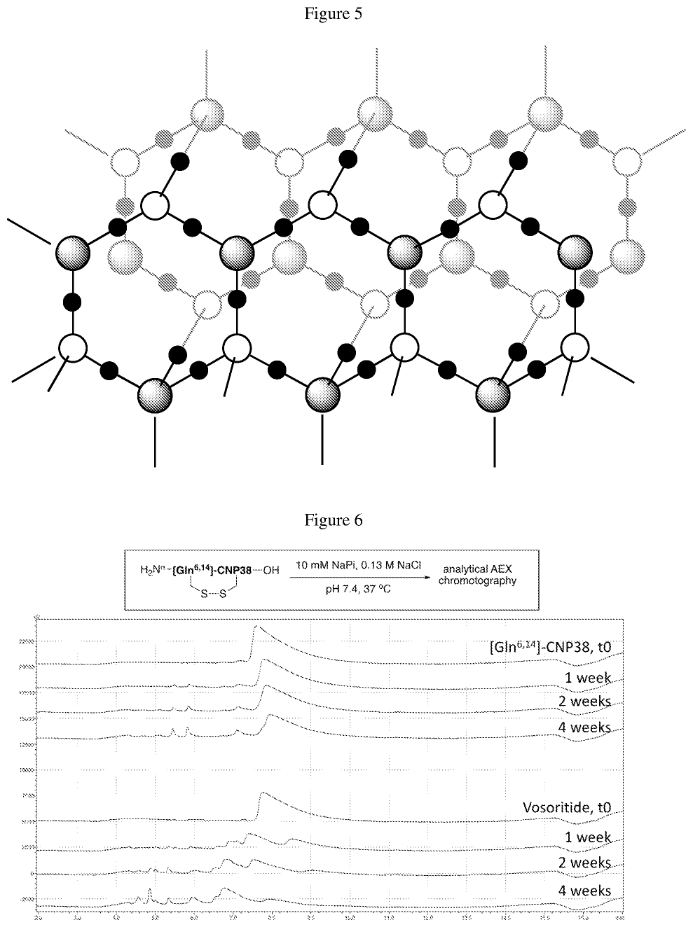

Exemplary structures of the crosslinks in such conjugates of Formula (IV) are given in FIG. 3. FIG. 2 further illustrates a method for prepared a conjugate of Formula (IV) comprising contacting a hydrogel of Formula (III) comprising connecting group Z′ with a linker-peptide of formula (II) comprising cognate connecting group Z, under conditions such that Z and Z′ react to form a residual group Z* that connects the linker-peptide to the hydrogel. When Z and Z′ are azide/cyclooctyne, such conditions are at a temperature between 0 and 100° C., preferably between 0 and 50° C., and more preferably between 25 and 50° C. The solvent in which the linker-peptide and hydrogel are suspended or dissolved may be aqueous, organic, or mixed depending on the solubility of the linker-peptide. Typical solvents are an aqueous buffer having a pH between 2 and 7, preferably between 2 and 5, optionally mixed with a water-miscible cosolvent such as methanol, ethanol, 2-propanol, tert-butanol, acetonitrile, dimethylformamide, acetonitrile, or tetrahydrofuran. The conjugates of formula (I) when M is an insoluble matrix are optionally isolated by washing to remove unreacted linker-peptide and reaction byproducts. Procedures for the reaction of (II) and (III) are analogous to those reported by Henise et al. (2020) Engineering Reports 2020; 2:e12213.

The linker-CNP of formula (II) may be prepared by reaction of a CNP peptide or protected version thereof with a linker reagent of formula (V) wherein X is a leaving group such as chloride, O-succinimidyl, O-nitrophenyl, and the like, and the remaining groups are as disclosed herein for formula (II).

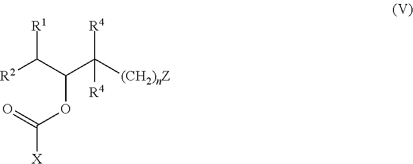

The linker may be attached to either the N-terminal alpha-amine or a lysine epsilon-amine group of the CNP peptide, using chemistry such as that described in U.S. Pat. No. 8,680,315. When the linker is attached to the N-terminal alpha-amine, this is preferably done during synthesis of the peptide on solid support as illustrated in FIG. 3. After synthesis of the protected peptide sequence on the solid support, the linker is attached by reaction with an active carbonate such as a succinimidyl carbonate as detailed in the Examples below, and the resulting intermediate is deblocked, cleaved from the resin, and the disulfide is formed to provide the linker-CNP of formula (II).

In a third aspect, the invention is directed to protocols for formulating and administering the conjugates of formula (I). In one embodiment, the conjugates are prepared as hydrogel microspheres suitable for subcutaneous injection using a narrow-gauge needle. These microspheres may be formulated in any solution suitable for injection and may comprise any excipients required to maintain the stability of the conjugate, for example buffers, antibacterial agents, antioxidant agents, agents for adjustment of density and osmolarity, and agents to promote suspension and prevent clumping of the microspheres. In particular, given the pH-sensitive nature of the beta-elimination mechanism for linker cleavage in these conjugates, stability is promoted by use of a low-pH buffer, preferably a buffer at pH 2-7, more preferably pH 3-7, and most preferably pH 4-5. Suitable buffers are those known in the art for pharmaceutical applications, and include acetate, citrate, malate, maleate, phosphate, and others effective in these pH ranges.

Administration can be by any route, including subcutaneous, intramuscular, or intra-articular. It is expected that the conjugates of the invention will be useful for the treatment of diseases and conditions in both humans and animals responsive to CNP, for example achondroplasia, with dosing frequencies of weekly, monthly, or longer.

All references cited herein are hereby incorporated by reference in their entireties. The following examples are provided to illustrate but not limit the scope of the invention.

The following examples will serve to illustrate rather than limit the scope of the disclosure. All references cited within are hereby incorporated by reference, including those cited for particular aspects of their disclosures, specifically for those aspects as well as in general.

### Preparation A

### Preparation of Linkers of Formula (V)

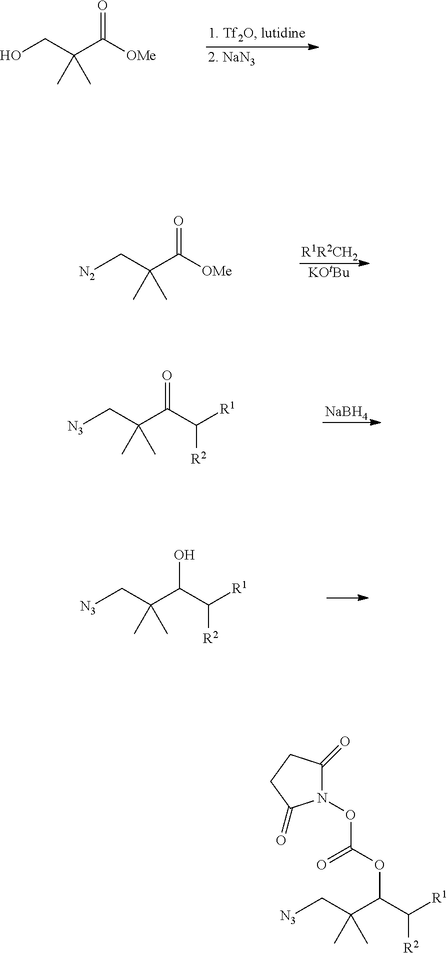

### 4-Azido-1-((N,N-dimethylamino)sulfonyl)-3,3-dimethyl-2-butyl succinimidyl carbonate

A 1.43 M solution of n-butyllithium in hexane (70 mL, 100 mmol) was added to a stirred solution of N,N-dimethyl methanesulfonamide (12.33 g, 100 mmol) in 200 mL of anhydrous THF kept at −50° C. under inert atmosphere. The mixture was allowed to warm to −20° C. over 1 h, then recooled to −50° C. before adding methyl 3-azido-2,2,-dimethylpropionate (prepared according to Kim, Synthetic Communications; 7.70 g, 50 mmol). The mixture was allowed to warm to +10° C. over 2 h, then quenched with 20 mL of 6 N HCl. The mixture was diluted with methyl t-butyl ether (MTBE, 200 mL), washed 2×100 mL of water and 1×100 mL of brine, dried over MgSO₄, filtered, and concentrated to yield 14.05 g of crude ketone product. Chromatography on SiO₂ (220 g) using a step gradient of 0, 20, 30, 40, and 50% EtOAc/hexane yielded purified 4-azido-1-((N,N-dimethylamino)sulfonyl)-3,3-dimethyl-2-butanone (10.65 g, 86%) as a crystalline solid.

The above ketone was dissolved in 200 mL of methanol, cooled on ice, and treated with sodium borohydride (0.96 g, 25 mmol) for 15 min before quenching with 4 mL of 6 N HCl and concentrating. The resulting slurry was diluted with methyl t-butyl ether (MTBE, 200 mL), washed 1×100 mL of water and 1×100 mL of brine, dried over MgSO₄, filtered, and concentrated to yield 10.0 g of crystalline 4-azido-1-((N,N-dimethylamino)sulfonyl)-3,3-dimethyl-2-butanol.

Pyridine (10.6 mL, 132 mmol) was added over 10 min to a stirred mixture of N-hydroxysuccinimide (6.90 g, 60 mmol) and triphosgene (5.93 g, 20 mmol) in 250 mL of dichloromethane cooled on ice. The mixture was stirred for 15 min on ice, then allowed to warm to ambient temperature over 30 min. A solution of 4-azido-1-((N,N-dimethylamino)sulfonyl)-3,3-dimethyl-2-butanol (10.0 g, 40 mmol) in 20 mL of dichloromethane was added and the mixture was stirred an additional 1 h at ambient temperature. After cooling on ice, the mixture was treated with 100 mL of water and the phases were separated. The organic phase was washed 2× water, 1×5% KHSO₄, and 1× brine, dried over MgSO₄, filtered, and concentrated. The crude product was crystallized from 100 mL of 30% EtOAc/hexane, providing 4-azido-1-((N,N-dimethylamino)sulfonyl)-3,3-dimethyl-2-butyl succinimidyl carbonate (11.1 g, 71%) as a white crystalline solid.

### 4-Azido-1-(isopropylsulfonyl)-3,3-dimethyl-2-butyl succinimidyl carbonate

A 1.38 M solution of n-butyllithium in hexanes (14.5 mL, 20 mmol) was added to a solution of diisopropylamine (2.96 mL, 21 mmol) in 80 mL of THF at −78° C., and the mixture was allowed to warm briefly to ambient temperature, then recooled to −78° C. Isopropyl methyl sulfone (2.69 g, 22 mmol) was added dropwise over 5 minutes, followed by methyl 3-azido-2,2,-dimethylpropionate (1.57 g, 10 mmol). The mixture was allowed to warm slowly to ambient temperature over 1 h, then quenched by addition of 3.47 mL of 6 N HCl (20.5 mmol). The mixture was diluted with ethyl acetate, washed 2×100 mL of water and 1×100 mL of brine, dried over MgSO₄, filtered, and concentrated to yield 2.9 g of crude ketone product as a yellow liquid. Chromatography on SiO₂ (25 g) using a gradient of 0-70% EtOAc/hexane yielded purified 4-azido-1-(isopropylsulfonyl)-3,3-dimethyl-2-butanone (1.73 g, 70%) as a crystalline solid.

The above ketone was dissolved in 14 mL of methanol, cooled on ice, and treated with sodium borohydride (0.13 g, 3.5 mmol) for 15 min before quenching with 1.2 mL of 6 N HCl and concentrating. The resulting slurry was diluted with methyl t-butyl ether (MTBE, 200 mL), washed 1×100 mL of water and 1×100 mL of brine, dried over MgSO₄, filtered, and concentrated to yield 1.51 g of 4-azido-1-(isopropylsulfonyl)-3,3-dimethyl-2-butanol as a colorless oil.

Pyridine (0.93 mL, 11.6 mmol) was added over 1 min to a stirred mixture of the alcohol (1.44 g, 5.78 mmol) and triphosgene (2.92 g, 9.83 mmol) in 45 mL of THF. After 30 min, the suspension was filtered and concentrated. The residue was redissolved in 25 mL of THF and treated with N-hydroxysuccinimide (1.97 g, 17.1 mmol) and pyridine (1.38 mL, 17.1 mmol), stirred for 30 min, then diluted with ethyl acetate was washed with 5% KHSO₄, water, and brine, dried with MgSO₄, filtered, and evaporated. Chromatography on SiO₂ (25 g) using a gradient of 0-80% EtOAc/hexane yielded purified 4-azido-1-(isopropylsulfonyl)-3,3-dimethyl-2-butyl succinimidyl carbonate (1.46 g) as a white crystalline solid. Linkers prepared according to these procedures include, without limitation:
4-Azido-1-(methylsulfonyl)-3,3-dimethyl-2-butyl succinimidyl carbonate (formula (V) wherein R¹=MeSO₂, R²=H, each R⁴=Me, Z=azide, X=O-succinimidyl, and n=1.
4-Azido-1-(isopropylsulfonyl)-3,3-dimethyl-2-butyl succinimidyl carbonate (formula (V) wherein R¹=ⁱPrSO₂, R²=H, each R⁴=Me, Z=azide, X=O-succinimidyl, and n=1.
4-Azido-1-(phenylsulfonyl)-3,3-dimethyl-2-butyl succinimidyl carbonate (formula (V) wherein R¹=PhSO₂, R²=H, each R⁴=Me, Z=azide, X=O-succinimidyl, and n=1.
4-Azido-1-(4-methylphenylsulfonyl)-3,3-dimethyl-2-butyl succinimidyl carbonate (formula (V) wherein R¹=(4-methylphenyl)SO₂, R²=H, each R⁴=Me, Z=azide, X=O-succinimidyl, and n=1.
4-Azido-1-(chlorophenylsulfonyl)-3,3-dimethyl-2-butyl succinimidyl carbonate (formula (V) wherein R¹=(4-chlorophenyl)SO₂, R²=H, each R⁴=Me, Z=azide, X=O-succinimidyl, and n=1.
4-Azido-1-cyano-3,3-dimethyl-2-butyl succinimidyl carbonate (formula (V) wherein R¹=CN, R²=H, each R⁴=Me, Z=azide, X=O-succinimidyl, and n=1.
4-Azido-1-(N,N-dimethylaminosulfonyl)-3,3-dimethyl-2-butyl succinimidyl carbonate (formula (V) wherein R¹=(Me₂N)SO₂, R²=H, each R⁴=Me, Z=azide, X=O-succinimidyl, and n=1.
4-Azido-1-(morpholinosulfonyl)-3,3-dimethyl-2-butyl succinimidyl carbonate (formula (V) wherein R¹=(O(CH₂CH₂)₂NSO₂, R²=H, each R⁴=Me, Z=azide, X=O-succinimidyl, and n=1.
4-Azido-1-(thiomorpholinosulfonyl)-3,3-dimethyl-2-butyl succinimidyl carbonate (formula (V) wherein R¹=(S(CH₂CH₂)₂NSO₂, R²=H, each R⁴=Me, Z=azide, X=O-succinimidyl, and n=1.
   Beginning with ethyl 4-chloro-2,2-dimethylbutyrate, the following linkers were prepared:
5-Azido-1-(chlorophenylsulfonyl)-4,4-dimethyl-2-pentyl succinimidyl carbonate (formula (V) wherein R¹=(4-chlorophenyl)SO₂, R²=H, each R⁴=Me, Z=azide, X=O-succinimidyl, and n=2.
5-Azido-1-cyano-4,4-dimethyl-2-pentyl succinimidyl carbonate (formula (V) wherein R¹=CN, R²=H, each R⁴=Me, Z=azide, X=O-succinimidyl, and n=2.
5-Azido-1-(phenylsulfonyl)-4,4-dimethyl-2-pentyl succinimidyl carbonate (formula (V) wherein R¹=phenylSO₂, R²=H, each R⁴=Me, Z=azide, X=O-succinimidyl, and n=2.
5-Azido-1-(methylsulfonyl)-4,4-dimethyl-2-pentyl succinimidyl carbonate (formula (V) wherein R¹=MeSO₂, R²=H, each R⁴=Me, Z=azide, X=O-succinimidyl, and n=2.

### Preparation B

### Hydrogels of Formula (III)

Hydrogels of formula (III) were prepared as sterile injectable microspheres as described in Henise et al (2020) Engineering Reports 2(8): e12213. Hydrogels prepared include those wherein:
(a) P¹ and P² are each 10-kDa 4-armed poly(ethylene glycol); B*=triazole; C*=carboxamide; q=1; R^{1a}=(N,N-dimethylamino)sulfonyl; R^{2a}=H; each R^{4a}=methyl; x=4; y=0; and Z′=NH—CO—O-(bicyclo[6.1.0]non-4-yn-9-ylmethyl).
(b) P¹ and P² are each 10-kDa 4-armed poly(ethylene glycol); B*=triazole; C*=carboxamide; q=1; R^{1a}=(N,N-dimethylamino)sulfonyl; R^{2a}=H; each R^{4a}=methyl; x=0; y=4; and Z′=NH—CO—O-(bicyclo[6.1.0]non-4-yn-9-ylmethyl).
(c) P¹ and P² are each 20-kDa 4-armed poly(ethylene glycol); B*=triazole; C*=carboxamide; q=1; R^{1a}=(N,N-dimethylamino)sulfonyl; R^{2a}=H; each R^{4a}=methyl; x=4; y=0; and Z′=NH—CO—O-(bicyclo[6.1.0]non-4-yn-9-ylmethyl).
(d) P¹ and P² are each 20-kDa 4-armed poly(ethylene glycol); B*=triazole; C*=carboxamide; q=1; R^{1a}=(N,N-dimethylamino)sulfonyl; R^{2a}=H; each R^{4a}=methyl; x=0; y=4; and Z′=NH—CO—O-(bicyclo[6.1.0]non-4-yn-9-ylmethyl).
(e) P¹ and P² are each 20-kDa 4-armed poly(ethylene glycol); B*=triazole; q=4; R^{1a}=CN; R^{2a}=H; each R^{4a}=H; x=0; y=4; and Z′=NH—CO—O-(4-cyclooctynyl). P¹ and P² are each 20-kDa 4-armed poly(ethylene glycol); B*=triazole; C*=carboxamide; q=1; R^{1a}=(N,N-dimethylamino)sulfonyl; R^{2a}=H; each R^{4a}=methyl; x=4; y=0; and Z′=NH—CO—O-(4-cyclooctynyl).
(f) P¹ and P² are each 20-kDa 4-armed poly(ethylene glycol); B*=triazole; C*=carboxamide; q=4; R^{1a}=CN; R^{2a}=H; each R^{4a}=H; x=0; y=4; and Z′=NH—CO—O-(4-cyclooctynyl).

### EXAMPLE 1

### Stability Study of (Gln^{6,14})CNP38 and Vosoritide

In 1.5 mL glass vials, 100 μM [Gln^{6,14}]-CNP38 or 100 μM vosoritide was treated with 10 mM sodium phosphate containing 0.13 M NaCl, pH 7.4. The reactions were kept at 37° C., and aliquots were periodically removed and stored at −20° C. prior to analysis. Samples were analyzed on a Dionex BioLC, ProPac SCX-10, 250×4 mm; 0-100% Buffer B over 10 min; 1 mL/min; 40° C. column temperature. Peaks were monitored at 220 nm. Buffer A: 10 mM MES, pH 6.2 @ 22° C.; Buffer B: 10 mM MES, 1 M NaCl, pH 6.2 @ 22° C. Significant decomposition of vosoritide was observed within 1 week while [Gln^{6,14}]-CNP38 was significantly more stable over the entire 4-week duration of the stability experiment (FIG. 6).

### EXAMPLE 2

### Preparation of Linker-CNP of Formula (II)

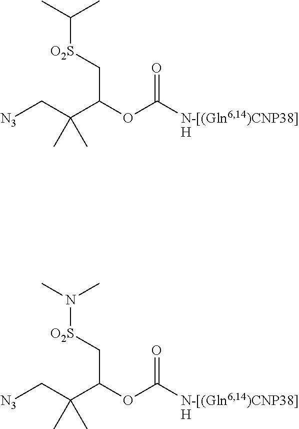

Linker-CNPs of Formula (II) were prepared by solid-phase peptide synthesis. Methods for preparing CNP with suppression of C-terminal cysteine racemization have been described in Fujiwara et al., Chem. Pharm. Bull. (1996) 44(7): 1326-31. Once the peptide sequence for (Gln^{6,14})CNP was prepared on resin, the linker was attached as the final residue using either 4-azido-3,3-dimethyl-1-(isopropylsulfonyl)-2-butyl succinimidyl carbonate or 4-azido-3,3-dimethyl-1-((N,N-dimethylamino)sulfonyl)-2-butyl succinimidyl carbonate. After disulfide formation, deblocking and cleavage from the resin, the linker-CNP was isolated by reversed-phase HPLC.

### EXAMPLE 3

### Preparation of CNP-Loaded Hydrogel Microspheres of Formula (IV)

Activated degradable hydrogel microspheres of Formula (III) wherein P¹ and P²=10-kDa 4-armed poly(ethylene glycols), B*=triazole, C*=carboxamide, q=1, R^{1a}=(N,N-dimethylamino)SO₂, R^{2a}=H, each R^{4a}=methyl, x=4, y=0, r=4, and Z′=(bicyclo[6.1.0]non-4-yn-9-yl)CH₂—O—CO—NH (“BCN-O—CO—NH”) were prepared as described in Henise et al. (2020) Engineering Reports 2020; 2:e12213. A slurry of the activated microspheres (2.7 mL of slurry, 15.0 μmol BCN) in reaction solvent (100 mM citrate in 1:1 iPrOH:H₂O at pH 3.0) was combined with the linker-CNP of Formula (II) wherein R¹=(N,N-dimethylamino)SO₂ (Example 2) at 1.2 equivalents to the BCN (4.8 μmol in 2.4 mL reaction solvent) and incubated at 37 C for 16 hours with agitation. The loaded microspheres were washed 4 times with 12 mL of the reaction solvent (until the OD₂₈₀ of the final wash was below detection) followed by 4 washes with 12 mL isotonic acetate buffer (10 mM Na acetate, 143 mM NaCl, 0.05% polysorbate 20 (w/v) pH 5.0. The CNP concentration and fraction loaded was determined by solubilizing ˜30 μL of the packed slurry (˜30 mg) in 9 volumes (˜270 μL, 1 μL:1 mg slurry) of 125 mM Borate pH 9.4 for 24 hours at 37° C. in duplicate. The peptide content was determined by absorbance at 276 nm (ε=5800 M⁻¹ cm⁻¹). The PEG content in each slurry was determined using the previously described PEG assay. The percent loading of the microsphere slurry was determined as the ratio of peptide concentration to the theoretical PEG reactive end groups based on the PEG assay. Similarly prepared was the conjugate wherein R¹=isopropyl-SO₂ (Example 2), using activated degradable hydrogel microspheres of Formula (III) wherein P¹ and P²=10-kDa 4-armed poly(ethylene glycols), B*=triazole, C*=carboxamide, q=1, R^{1a}=(N,N-dimethylamino)SO₂, R^{2a}=H, each R^{4a}=methyl, x=0, y=4, r=4, and Z′=(bicyclo[6.1.0]non-4-yn-9-yl)CH₂—O—CO—NH (“BCN-O—CO—NH”).

### EXAMPLE 4

### Kinetics of Peptide Release

Samples of the loaded microspheres (˜30 mg) were placed in 1.5 mL screw top microcentrifuge tubes and peptide release was initiated by addition of 9 volumes (—0.27 mL) of 100 mM Na Borate buffer pH 9.4. The reactions were incubated in a water bath at 37° C. At t=0 and various timepoints, the microsphere slurries were pelleted at 10,000×g, 5 μL of the reaction supernatant was removed and the samples were stored at −20° C. The concentration of (Gln^{6,14})CNP38 in the reaction supernatant was determined by absorbance at 280 nm on a Nanodrop UV-Vis. The Also of the supernatant timepoints were plotted and fit to a single exponential using Prism 8.0 software to determine the release rate for each peptide. For R¹=isopropyl-SO₂, a release half-life of 6.1 h was observed, corresponding to a release half-life of 610 h at pH 7.4, 37° C. For R¹=(N,N-dimethylamino)-SO₂, a release half-life of 21 h was observed, corresponding to a release half-life of 2100 h at pH 7.4, 37° C.

### EXAMPLE 5

### Pharmacokinetics Study in Mice

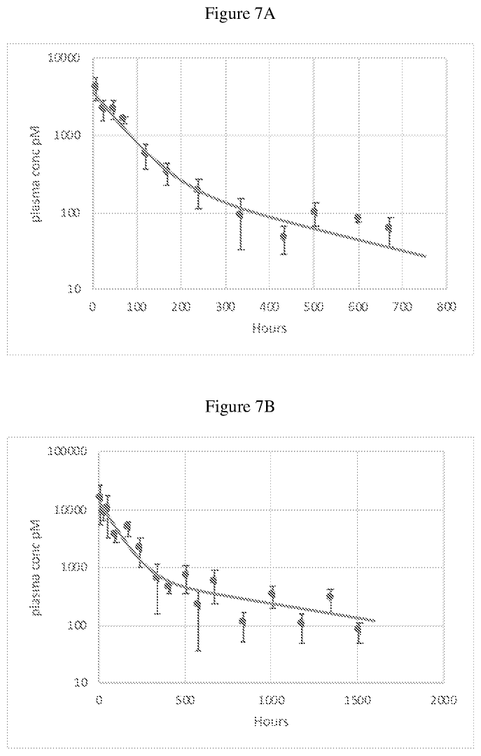

CD-1 mice (male, 7 week old, ˜30 g weight) were dosed by subcutaneous injection of a slurry of the hydrogel microspheres of Example 3 suspended in pH 5.0 buffer containing 10 mM sodium acetate, 143 mM NaCl, 10 mM methionine, 0.05% polysorbate 20 (w/v), and 1.2% sodium hyaluronate 60 kDa (w/v). The hydrogel microsphere conjugate wherein R¹=(N,N-dimethylamino)SO₂ was dosed at 0.7 μmol per mouse. Serum samples were collected with the addition of HALT protease inhibitors at the following timepoints: 0, 8, 24, 48, 96, 168, 240, 336, 408, 04, 576, 672, 840, 1008, 1176, 344, 1512, 1680, 1848, and 2016 hr. The complete time course was collected using 2 groups of 4 mice each and collecting serum samples at alternating timepoints from each group. The hydrogel microsphere conjugate wherein R¹=isopropyl-SO₂ was dosed at 0.22 μmol per mouse. Serum samples were collected with the addition of HALT protease inhibitors at the following timepoints: 0, 8, 24, 48, 72, 120, 168, 240, 336, 432, 504, 600, and 672 hr. The complete time course was collected using 3 groups of 4 mice each and collecting serum samples at every third timepoint from alternating groups. Serum samples were analyzed by ELISA, using (Gln^{6,14})CNP38 as a standard quantitated by absorbance (ε₂₈₀=1681.8 M⁻¹ cm⁻¹). Results are given in FIG. 7. Data were fit to a two-phase model. For R¹=isopropyl-SO₂, half-lives of 39 and 212 h were observed. For R¹=(N,N-dimethylamino)-SO₂, half-lives of 58 and 607 h were observed.

### EXAMPLE 6

### Pharmacokinetics in Cynomolgous Monkeys

Microspheres loaded with [Gln^{6,14}]CNP-38 were prepared as described in Example 3 and contained 3.1 and 3.6 umol peptide/mL packed slurry for R¹=isopropylsulfonyl and R¹=(N,N-dimethylamino)sulfonyl, respectively. The microspheres were formulated in isotonic acetate (10 mM Na Acetate, 143 mM NaCl) pH 5.0, 0.05% Tween 20 buffer containing 1.2% sodium hyaluronate. Syringes (0.3 mL U-100 insulin syringe with fixed 29 g×½″ needle, BD #34702) were filled under sterile conditions with 1 mL of formulated microspheres.

Normal, juvenile cynomolgus monkeys between 1.8 and 2.6 kg, aged >18 months at the onset of the experiment (3 per group, 1 female and 2 males each) were administered the conjugates of Example 3 subcutaneously at 1.3 μmol/kg (R¹=isopropylsulfonyl) or 1.2 μmol/kg (R¹=(N,N-dimethylamino)-sulfonyl) based on the current weight. Any unneeded material was expelled from the prefilled syringes and the remaining volume dosed. Blood samples (approximately 1 mL) were collected from the peripheral vein of monkeys administered 4A and 4B at predose, 8, 24, 48, 96, 168, 240, 336, 408, 504, 576, 672, 744, and 840 hours after dose administration. For monkeys administered 4B, blood was also collected at 1008, 1176, 1344, 1680, and 2160 hours after dose administration. Blood samples were processed to plasma by collection in tubes containing K₂EDTA and protease inhibitor (Halt protease inhibitor, ThermoFisher Scientific) and split into two equal volumes for storage at −80 C. Beginning on Day 1 after the first dose, the animals were examined for general signs of toxicity, including fecal and urine quality, at least once weekly at the injection site, head, neck, limbs, trunk, tail, body orifices and genitalia. While handling, each animal was observed for changes in skin, fur, eyes, mucous membranes, occurrence of secretions and excretions, lacrimation, piloerection, pupil size, and unusual respiratory pattern. The animals' cages were also inspected for abnormal feces, urine, vomitus or other excretions or secretions.

[Gln^{6,14}]CNP-38 concentrations in cynomolgus plasma were measured using the CNP-22 fluorescent EIA Kit from Phoenix Pharmaceuticals, Inc (cat #FEK-012-03) read on a Molecular Devices Spectramax i3 plate reader. Plasma samples were diluted in blank cynomolgus monkey plasma to obtain concentrations in the range of a standard curve (2 pM to 2 nM) generated using [Gln^{6,14}]CNP-38 in place of CNP-22. Analysis was repeated by two separate operators on separate days using the two different sample aliquots.

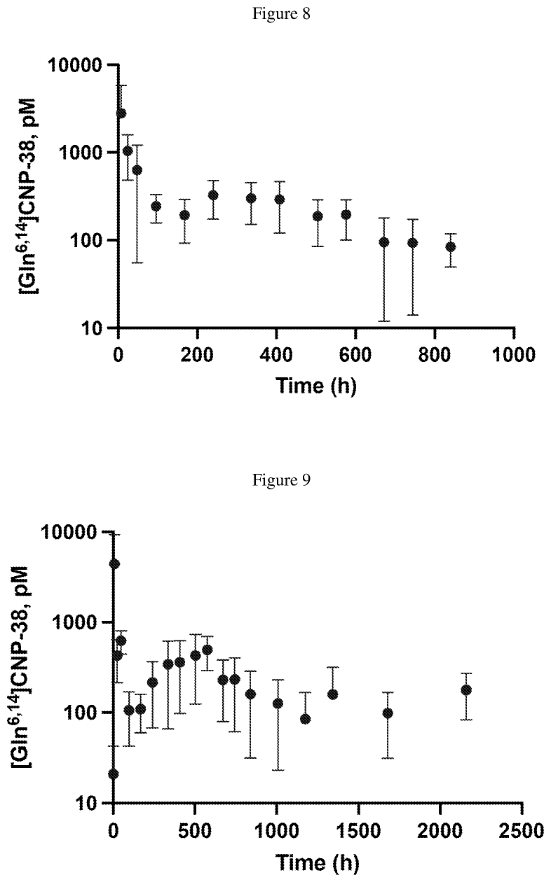

FIGS. 8 and 9 show the plasma concentrations of juvenile cynomolgous monkeys treated with Example 3, R¹=isopropylsulfonyl, in FIG. 8, or with Example 3, R¹=(N,N-dimethylamino)-sulfonyl, in FIG. 9. The conjugate of Example 3, wherein R¹=isopropylsulfonyl, provided continuous exposure ≥100 pM of [Gln^{6,14}]CNP-38 for approximately 1 month. The conjugate of Example 3, wherein R¹=(N,N-dimethylamino)-sulfonyl, provided continuous exposure ≥100 pM of [Gln^{6,14}]CNP-38 for greater than 3 months.

### EXAMPLE 7

### Pharmacodynamics in Juvenile Mice

Three-week old wild-type male mice (FVB/nJ; Charles River Laboratory, Inc) were administered subcutaneous injections of the conjugates of Example 3 at either a weight-adjusted (nmol/kg) or constant (nmol/animal independent of the initial weight) dose over 35 days. For experiments using constant doses/animal, the conjugate of Example 3 wherein R¹=isopropylsulfonyl (conjugate 4A) was administered at either 20 or 50 nmol/mouse; the conjugate of Example 3 wherein R¹=(N,N-dimethylamino)sulfonyl (conjugate 4B) was administered at either 85 or 600 nmol/mouse. For experiments using weight-adjusted doses/animal, conjugate 4A was administered at either 1.5 or 2.2 umol/kg; conjugate 4B was administered at 6.1 umol/kg. Doses were calculated based on the body weight just prior to dosing and performed at approximately the same time of day. Comparator animals were dosed with either [Gln^{6,14}]CNP-38 or vosoritide at 70 nmol/kg formulated in 30 mM acetic acid pH 4.0 containing 10% (w/v) sucrose and 1% (v/v) benzyl alcohol (Wendt; Breinholt), and control animals were dosed with the formulation buffer (isotonic acetate (10 mM Na Acetate, 143 mM NaCl), pH 5.0, 0.05% Tween 20 buffer containing 1.2% sodium hyaluronate. Daily weight-adjusted doses were calculated based on the body weight just prior to dosing and performed at approximately the same time each day.

Blood collections were made via tail vein with the addition of protease inhibitors (Halt protease inhibitor cocktail, ThermoScientific) and processed with K₂EDTA to provide ˜15-20 μL plasma, for analysis of free [Gln^{6,14}]CNP-38. For the mice treated with the conjugate 4A, blood collection occurred at 0, 24, 168, 192, 336, 360, 504, 528, 672 and 840 hr. For the mice treated with conjugate 4B, blood collection occurred at 0, 24, 168, 336, 504, 672 and 840 hr.

Body weight, tail length and naso-anal length were collected at study initiation and were measured weekly during the in-life treatment period. The mice were anesthetized using isoflurane and placed on a table in the supine position, the tail was pulled straight, and a metal ruler was used to gently press down on the mouse to straighten any curvature of the spine so that the mouse was fully elongated. The naso-anal (distance from the tip of the nose to the anus) and the tail length (distance from the anus to the tip of the tail) were measured and recorded. The same ruler was used for all measurements, and all measurements were conducted and recorded in centimeters. Total length (TL) is derived from the addition of the measured tail length and naso-anal length.

On day 35, the mice were anesthetized and in addition to the nasal-anal and tail length measurements, bone length measurements were made by digital X-ray (spine, right femur, right tibia, humerus and ulna). Following deep anesthesia with isoflurane, the mice were placed in the prone position and the length of the spine (lateral view, distance from the most cranial end of the C1 vertebra to the most caudal end of the S4 vertebra and accounting for curvature manually), the right femur (distance from the most proximal femoral head ossification center to the most distal ossification center in dorso-ventral view), the right tibia (distance from the most proximal ossification center to the most distal ossification center in dorso-ventral view), the right humerus (distance from the most proximal ossification center to the most distal ossification center in dorso-ventral view), and the right ulna (distance from the most proximal ossification center to the most distal ossification center in dorso-ventral view) of each mouse were measured by x-ray using a Spectral Imaging Instruments AMI HTX and the associated Aura software.

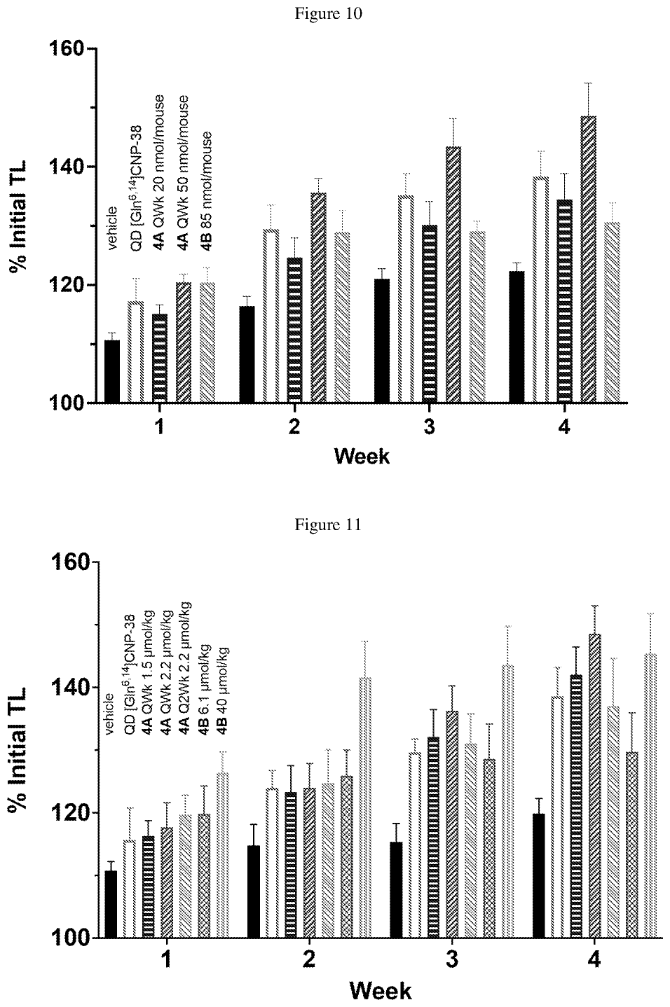

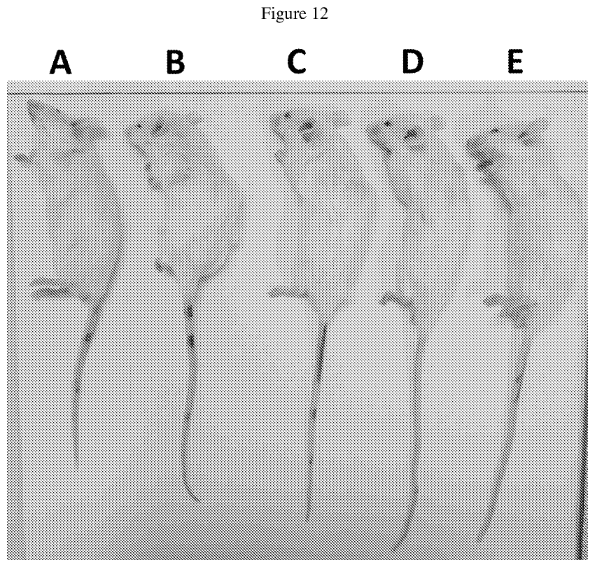

The total tail length measurements for the weight-adjusted dosing are shown in FIGS. 10 and 11, respectively. FIG. 12 shows photograph of the anesthetized mice treated with A) vehicle control; B) QD [Gln^{6,14}]CNP-38 peptide at 70 nmol; C) biweekly (Q2Wk) conjugate 4A at 2.2 μmol/kg; D) weekly (QWk) conjugate 4A at 2.2 μmol/kg; and E) weekly (QWk) conjugate 4A at 1.5 μmol/kg, after five weeks.

Table 1 shows bone length after 5 weeks of treatment with the conjugates of Example 3 wherein R¹=isopropylsulfonyl (4A) or R¹=(N,N-dimethylamino)sulfonyl (4B). Measured by X-ray. [standard deviation] (% change from vehicle).

The results showed that a weekly (QWk) dose of 4A containing 1.5 μmol/kg [Gln^{6,14}]CNP-38, or biweekly (Q2Wk) dose containing 2.2 μmol/kg [Gln^{6,14}]CNP-38 increased growth relative to vehicle control comparable to or exceeding that of daily (QD) [Gln^{6,14}]CNP-38 or vosoritide. A single dose of conjugate 4B at 6.1 μmol/kg also supported increased growth comparable to daily [Gln^{6,14}]CNP-38 for at least 3 weeks before stabilizing. These results suggest appropriate regimens of long-acting 4A and 4B delivered weekly, biweekly or monthly can achieve similar or greater growth enhancement of juvenile mice as daily dosing of vosoritide or other CNP variants.

TABLE 1

| | | Bone Length (mm) | | | | |
|---|---|---|---|---|---|---|
| Treatment | Dose | Femur | Tibia | Spine | Humerus | Ulna |
| vehicle | N/A | 12.5 [0.7] | 18.4 [0.2] | 57.5 [1.2] | 12.2 [0.5] | 14.8 [0.1] |
| [Gln6, 14] | QD 70 | 14.2 [1.0] | 20.0 [0.5] | 63.3 [1.4] | 12.3 [0.8] | 15.6 [0.4] |
| CNP-38 | nmol/kg | (13.6%) | (8.7%) | (10.1%) | (0.8%) | (5.4%) |
| 4A | QWk 50 | 14.1 [1.0] | 21.9 [0.3] | 74.1 [2.5] | 13.9 [0.9] | 16.3 [0.4] |
| | nmol/mouse | (12.8%) | (19.0%) | (28.9%) | (13.9%) | (10.1%) |
| | Qwk 20 | 13.5 [0.9] | 20.2 [0.9] | 64.9 [0.9] | 12.7 [0.6] | 16.0 [0.4] |
| | nmol/mouse | (8.0%) | (9.8%) | (12.9%) | (4.1%) | (8.1%) |
| | QWk 1.5 | 16.1 [0.4] | 21.1 [0.8] | 71.2 [1.4] | 13.1 [0.9] | 16.7 [0.5] |
| | μmol/kg | (28.8%) | (14.7%) | (26.0%) | (4.8%) | (12.1%) |
| | QWk 2.2 | 15.5 [1.3] | 20.9 [1.0] | 73.8 [2.6] | 13.7 [0.8] | 16.8 [0.5] |
| | μmol/kg | (24.0%) | (13.6%) | (30.6%) | (9.6%) | (12.8%) |
| | Q2Wk 2.2 | 14.5 [0.8] | 20.4 [0.6] | 66.4 [1.7] | 13.4 [0.7] | 16.0 [0.6] |
| | μmol/kg | (16.0%) | (10.9%) | (17.5%) | (7.2%) | (7.4%) |
| 4B | s.d. 84 | 12.7 [1.1] | 19.3 [0.4] | 62.0 [1.8] | 12.5 [0.2] | 14.8 [0.5] |
| | nmol/mouse | (1.6%) | (4.9%) | (7.8%) | (2.5%) | (0.0%) |
| | s.d. 6.1 | 14.5 [1.5] | 19.2 [0.8] | 61.9 [3.2] | 12.8 [0.7] | 15.6 [0.5] |
| | μmol/kg | (16.0%) | (4.3%) | (9.6%) | (2.4%) | (4.7%) |
| | s.d. 40 | 11.7 [0.8] | 20.3 [0.5] | 64.0 [3.8] | 12.1 [0.9] | 15.9 [0.5] |
| | μmol/kg | (−6.4%) | (10.3%) | (11.3%) | (−0.8%) | (7.4%) |

### EXAMPLE 8

### Kinetics of Peptide Release and Hydrogel Degradation

Biodegradable hydrogel microspheres loaded with (Gln^{6,14})CNP38 prepared according to Example 3 (R¹=isopropylsulfonyl or (N,N-dimethylamino)sulfonyl) were analyzed for release of free (Gln^{6,14})CNP38 and dissolved PEG under accelerated conditions of pH 9.4, 37° C., according to the published procedures (Henise et al. (2020) Engineering Reports 2020; 2:e12213).

Solubilized peptide was quantitated by OD₂₈₀ while solubilized PEG was quantitated using a BaCl₂/I₂/NaI colorometric assay using PEG8000 as standard. As some early degradation of the hydrogel microspheres may result in dissolution of PEG fragments that still have peptide attached, the total solubilized peptide measured by OD₂₈₀ may be the sum of free peptide released from the microspheres plus solubilized PEGylated peptide. The data were thus analyzed by correcting the amount of soluble peptide, assuming a constant first-order rate of cleavage of the drug-linker. Assuming that the solubilized PEG is representative of the bulk hydrogel, then the amount of soluble PEG-peptide contributing to the total OD280 is simply given as

(PEG-peptide)ₛₒₗ=PEG(t)·exp(−kt)

where PEG(t)=the amount of solubilized PEG present at time t and k=the first-order rate constant for linker-peptide cleavage. This amount is subtracted from the total peptide measured by OD280 to give the amount of free peptide released from the hydrogel at time t. The experimental data for OD280 and PEG were normalized according to the totals in the assay as determined by the plateau values, and then the normalized data were fit using an iterative process to determine the value of k giving the best fit according to the sum of squared residuals.

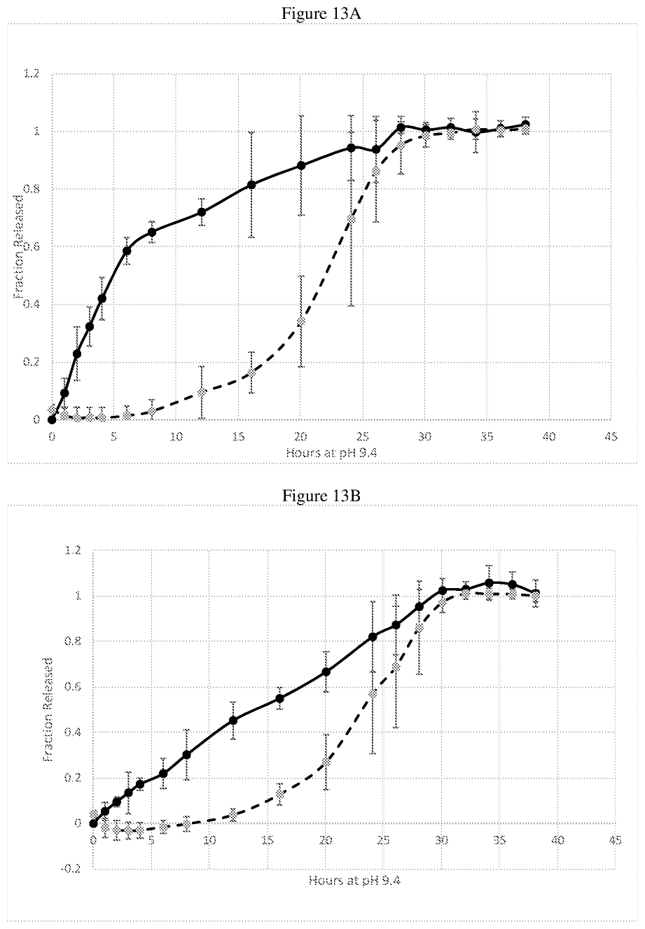

Peptide release and hydrogel degradation were measured using 6 replicates, with normalized values for OD₂₈₀ and solubilized PEG being averaged. The results are shown in FIGS. 13A and 13B.

For the conjugate of Example 3 wherein R¹=isopropylsulfonyl, as shown in FIG. 13A, a best-fit value fork gave a t_{1/2}=6.1 h at pH 9.4, which extrapolates to 610 h at pH 7.4, 37° C. The hydrogel microspheres were completely dissolved by ˜30 h at this pH. For the conjugate wherein R¹=(N,N-dimethylamino)sulfonyl, as shown in FIG. 13B, a best-fit value for k gave a t_{1/2}=15.8 h at pH 9.4, which extrapolates to 1580 h at pH 7.4, 37° C. The hydrogel microspheres were completely dissolved by ˜30 h at this pH.

1. A hydrogel conjugate of a C-natriuretic peptide having formula (I)
M-(L-E)ₐ (I)
wherein M is a hydrogel matrix;
L is a linker;
E is a C-natriuretic peptide; and
a is an integer that represents the number of L-E moieties that yield a suitable concentration of E in a given volume of the matrix.

2. The hydrogel conjugate of claim 1, wherein E is selected from the group consisting of SEQ ID Nos: 1-6.

3. The hydrogel conjugate of claim 1, wherein E is (Gln^{6,14})CNP38 (SEQ ID No: 4).

4. The hydrogel conjugate of claim 1, wherein M is a biodegradable hydrogel of formula (III) 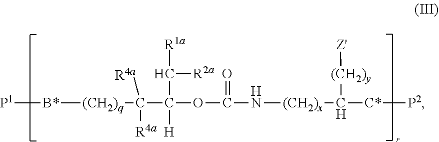
wherein
q=0-6;
R^{1a} and R^{2a} are independently H, alkyl, CN, NO₂, optionally substituted aryl, optionally substituted heteroaryl, optionally substituted alkenyl, optionally substituted alkynyl, —COR^{3a}, —SOR^{3a}, or —SO₂R^{3a}, wherein
R^{3a} is H, optionally substituted alkyl, optionally substituted aryl, optionally substituted arylalkyl, optionally substituted heteroaryl, optionally substituted heteroarylalkyl, —OR^{5a}, or —NR^{5a}₂, wherein
each R^{5a} is independently H or optionally substituted alkyl, or both R^{5a} groups are taken together with the nitrogen to which they attach to form a heterocyclic ring;
wherein R^{1a} and R^{2a} may be taken together with the carbon to which they attach to form a 3-8 membered ring, and wherein one and only one of R^{1a} and R^{2a} may be H or alkyl;
each R^{4a} is independently H or C₁-C₃ alkyl, or both R^{4a} are taken together with the carbon to which they attach to form a 3-6 membered ring;
Z′ is a functional group for mediating coupling to linker -L-E;
x and y are each independently 0-6;
B* and C* are each independently a connecting group; and
P¹ and P² are independently r-armed polymers of 1-40 kDa average molecular weight, wherein r is an integer from 2 to 8.

5. The hydrogel conjugate of claim 1, wherein L comprises a residue having the formula 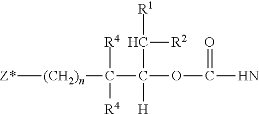
wherein:
n=0-6;
R¹ and R² are independently H, alkyl, CN, NO₂, optionally substituted aryl, optionally substituted heteroaryl, optionally substituted alkenyl, optionally substituted alkynyl, —COR³, —SOR³, or —SO₂R³, wherein
R³ is H, optionally substituted alkyl, optionally substituted aryl, optionally substituted arylalkyl, optionally substituted heteroaryl, optionally substituted heteroarylalkyl, —OR⁵, or —NR⁵₂, wherein
each R⁵ is independently H or optionally substituted alkyl, or both R⁵ groups are taken together with the nitrogen to which they attach to form a heterocyclic ring;
wherein R¹ and R² may be taken together with the carbon to which they attach to form a 3-8 membered ring, and wherein one and only one of R¹ and R² may be H or alkyl;
each R⁴ is independently H or C₁-C₃ alkyl, or both R⁴ are taken together with the carbon to which they attach to form a 3-6 membered ring;
Z* is a connecting group; and
NH is the residue of an amino group of a CNP peptide.

6. The hydrogel conjugate of claim 5, wherein R¹ is CN or SO₂R³.

7. The hydrogel conjugate of claim 5, wherein each R⁴ is independently C₁-C₃ alkyl, or both R⁴ are taken together with the carbon to which they attach to form a 3-6 membered ring.

8. The hydrogel conjugate of claim 1, wherein the hydrogel conjugate is the formula (IV) 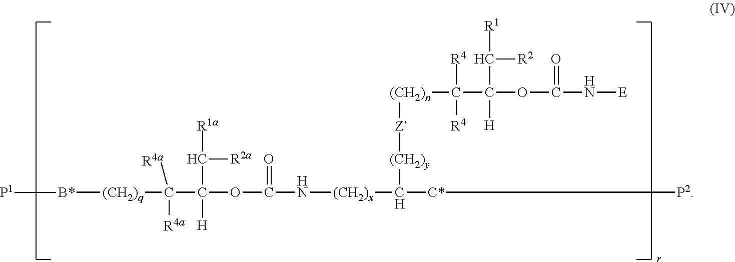
wherein
n=0-6;
R¹ and R² are independently H, alkyl, CN, NO₂, optionally substituted aryl, optionally substituted heteroaryl, optionally substituted alkenyl, optionally substituted alkynyl, —COR³, —SOR³, or —SO₂R³, wherein
R³ is H, optionally substituted alkyl, optionally substituted aryl, optionally substituted arylalkyl, optionally substituted heteroaryl, optionally substituted heteroarylalkyl, —OR⁵, or —NR⁵₂, wherein
each R⁵ is independently H or optionally substituted alkyl, or both R⁵ groups are taken together with the nitrogen to which they attach to form a heterocyclic ring;
wherein R¹ and R² may be taken together with the carbon to which they attach to form a 3-8 membered ring, and wherein one and only one of R¹ and R² may be H or alkyl;
each R⁴ is independently H or C₁-C₃ alkyl, or both R⁴ are taken together with the carbon to which they attach to form a 3-6 membered ring;
Z*, B*, and C* are each independently a connecting group;
E is a CNP peptide;
q=0-6;
R^{1a} and R^{2a} are independently H, alkyl, CN, NO₂, optionally substituted aryl, optionally substituted heteroaryl, optionally substituted alkenyl, optionally substituted alkynyl, —COR^{3a}, —SOR^{3a}, or —SO₂R^{3a}, wherein
R^{3a} is H, optionally substituted alkyl, optionally substituted aryl, optionally substituted arylalkyl, optionally substituted heteroaryl, optionally substituted heteroarylalkyl, —OR^{5a}, or —NR^{5a}₂, wherein
each R^{5a} is independently H or optionally substituted alkyl, or both R^{5a} groups are taken together with the nitrogen to which they attach to form a heterocyclic ring;
wherein R^{1a} and R^{2a} may be taken together with the carbon to which they attach to form a 3-8 membered ring, and wherein one and only one of R^{1a} and R^{2a} may be H or alkyl;
each R^{4a} is independently H or C₁-C₃ alkyl, or both R^{4a} are taken together with the carbon to which they attach to form a 3-6 membered ring;
x and y are each independently 0-6; and
P¹ and P² are independently r-armed polymers of 1-40 kDa average molecular weight, wherein r is an integer from 2 to 8.

9. The hydrogel conjugate of claim 8, wherein R¹ is CN or SO₂R³ and R^{1a} is CN or SO₂R^{3a}.

10. The hydrogel conjugate of claim 8, wherein R^{1a} is CN or SO₂R³; R² is H; each R⁴ is methyl; n=1-2; E is SEQ ID No: 4; Z* and B* are triazole; C* is carboxamide; x=0-4; y=0-4; R^{1a} is CN or SO₂R^{3a}; R^{2a} is H; each R^{4a} is methyl; q=1-2; P¹ and P² are each independently 1-40 kDa r-armed poly(ethylene glycols); and r=4-8.

11. The hydrogel conjugate of claim 10, wherein R¹ is SO₂R³, wherein R³ is optionally substituted alkyl.

12. The hydrogel conjugate of claim 10, wherein R¹ is SO₂R³, wherein R³ is (R⁵)₂N.

13. A method of preparing the hydrogel conjugate of claim 1, comprising contacting a linker-CNP peptide of formula (II), 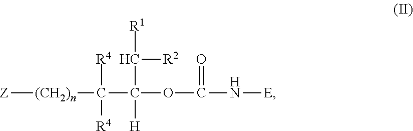
wherein:
n=0-6;
R¹ and R² are independently H, alkyl, CN, NO₂, optionally substituted aryl, optionally substituted heteroaryl, optionally substituted alkenyl, optionally substituted alkynyl, —COR³, —SOR³, or —SO₂R³, wherein
R³ is H, optionally substituted alkyl, optionally substituted aryl, optionally substituted arylalkyl, optionally substituted heteroaryl, optionally substituted heteroarylalkyl, —OR⁵, or —NR⁵₂, wherein
each R⁵ is independently H or optionally substituted alkyl, or both R⁵ groups are taken together with the nitrogen to which they attach to form a heterocyclic ring;
wherein R¹ and R² may be taken together with the carbon to which they attach to form a 3-8 membered ring, and wherein one and only one of R¹ and R² may be H or alkyl;
each R⁴ is independently H or C₁-C₃ alkyl, or both R⁴ are taken together with the carbon to which they attach to form a 3-6 membered ring;
Z is a functional group for mediating coupling to a hydrogel of formula (III) through a cognate functional group Z′; and
NH is the residue of an amino group of CNP peptide E,
with an activated hydrogel of formula (III), 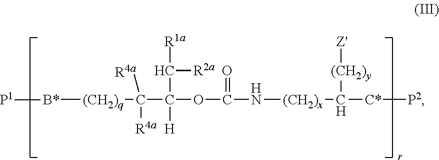
wherein q=0-6;
R^{1a} and R^{2a} are independently H, alkyl, CN, NO₂, optionally substituted aryl, optionally substituted heteroaryl, optionally substituted alkenyl, optionally substituted alkynyl, —COR^{3a}, —SOR^{3a}, or —SO₂R^{3a}, wherein
R^{3a} is H, optionally substituted alkyl, optionally substituted aryl, optionally substituted arylalkyl, optionally substituted heteroaryl, optionally substituted heteroarylalkyl, —OR^{5a}, or —NR^{5a}₂, wherein
each R^{5a} is independently H or optionally substituted alkyl, or both R^{5a} groups taken together with the nitrogen to which they attach to form a heterocyclic ring;
wherein R^{1a} and R^{2a} may be taken together with the carbon to which they attach to form a 3-8 membered ring, and wherein one and only one of R^{1a} and R^{2a} may be H or alkyl;
each R^{4a} is H or C₁-C₃ alkyl, or both R^{4a} are taken together with the carbon to which they attach to form a 3-6 membered ring;
Z′ is a functional group for mediating coupling to cognate functional group Z;
x and y are each independently 0-6;
B* and C* are each independently a connecting group; and
P¹ and P² are independently r-armed polymers of 1-40 kDa average molecular weight, wherein r is an integer from 2 to 8,
under conditions wherein cognate functional groups Z and Z′ react to form connecting group Z*, and optionally isolating the resulting conjugate.

14. A pharmaceutical composition comprising the hydrogel conjugate of claim 1 and a pharmaceutically acceptable buffer, wherein the pharmaceutical composition has a pH between 3 and 7.

15. A method of treating a disease or condition requiring treatment with a CNP peptide in a patient in need of such treatment, comprising administering the pharmaceutical composition of claim 14.

16. A C-natriuretic peptide, wherein the peptide is (Gln^{6,14})CNP38 (SEQ ID No: 4).
